(19) United States
(12) Patent Application Publication
(10) Pub. No.: US 2021/0311022 A1
(43) Pub. Date: Oct. 7, 2021

(54) METHODS, KITS AND COMPOSITIONS FOR CHARACTERIZING AN ANTI-INFLAMMATORY RESPONSE OF A PRODUCT

(71) Applicant: ORGANOGENESIS, INC., Birmingham, AL (US)

(72) Inventors: Katie C. MOWRY, Birmingham, AL (US); Kelly A. KIMMERLING, Hoover, AL (US); Miranda D. BURNETTE, Birmingham, AL (US); Mary Rose KAMMER, Birmingham, AL (US)

(21) Appl. No.: 17/215,921

(22) Filed: Mar. 29, 2021

Related U.S. Application Data
(60) Provisional application No. 63/001,005, filed on Mar. 27, 2020.
(60) Provisional application No. 63/164,846, filed on Mar. 23, 2021.

(51) Int. Cl. G01N 33/50; C12N 5/077

(52) U.S. Cl. CPC G01N 33/5044; G01N 2800/102; G01N 2333/5406; G01N 2333/5412; G01N 2333/5428; G01N 2333/5431; G01N 2333/5437; G01N 2333/50; G01N 2333/57; G01N 2333/495; G01N 2333/65; G01N 2333/51; G01N 2333/4704; C12N 2501/2301; C12N 2501/25; C12N 5/0652

(57) ABSTRACT

Disclosed are methods for methods for quantitating and standardizing an anti-inflammatory response of a product and methods for determining the likelihood that a product would produce an anti-inflammatory effect in a subject when administered to the subject. Accordingly, disclosed herein are methods, compositions and kits for characterizing and evaluating the anti-inflammatory effect of products, particularly therapeutic biological products.

### CROSS REFERENCE TO RELATED APPLICATIONS

This application claims priority to U.S. Provisional Patent Application No. 63/001,005, entitled “Methods, Kits, and Compositions for Characterizing an Anti-Inflammatory Response of a Product” and filed on Mar. 27, 2020, and U.S. Provisional Patent Application No. 63/164,846, entitled “Methods, Kits, and Compositions for Characterizing an Anti-Inflammatory Response of a Product” and filed on Mar. 23, 2021, which are incorporated herein by reference.

### BACKGROUND

Joint disease is a degenerative disease that involves the soft tissues, cartilage, and subchondral bones of the joints. Two exemplary forms of joint disease are rheumatoid arthritis (RA) and osteoarthritis (OA). The irreversible destruction of the cartilage, tendon, and bone that comprise synovial joints is the hallmark of both RA and OA. OA affects over 30 million Americans. RA and OA are slowly progressive diseases involving biomechanical, biochemical, and genetic factors. These pathways may each contribute to RA and OA lesion in cartilage by disrupting chondrocyte-matrix associations and altering metabolic responses in the chondrocyte. In early disease, the chondrocyte exhibits a transient proliferative response, increased synthesis of cartilage matrix as an early attempt at repair, and increased synthesis of catabolic cytokines and matrix degrading enzymes (including matrix metalloproteases (MMPs), aggrecanase, PA, cathepsins). Local loss of proteoglycans and cleavage of type II collagen occur initially at the cartilage surface resulting in an increase in water content and loss of tensile strength in the cartilage matrix as the lesion progresses. The central role of cytokines, particularly the pro-inflammatory cytokines interleukin (IL)-1β and tumor necrosis factor (TNF)-α, in causing the destruction of articular cartilage is well established in the art. Biologically, the pro-inflammatory cytokines IL-1β and TNF-α, along with the MMPs are responsible for the joint degradation observed.

While cartilage is made up of proteoglycans and type II collagen, tendon and bone are composed primarily of type I collagen. RA is an autoimmune disease afflicting numerous joints throughout the body; in contrast, OA develops in a small number of joints, usually resulting from chronic overuse or injury. In both diseases, inflammatory cytokines such as IL-1β and TNF-α, stimulate the production of MMPs, enzymes that can degrade all components of the extracellular matrix. MMP-1 and MMP-13 have predominant roles in RA and OA because they are rate limiting in the process of collagen degradation. MMP-1 is produced primarily by the synovial cells that line the joints, and MMP-13 is a product of the chondrocytes that reside in the cartilage. Expression of other MMPs such as MMP-2, MMP-3 and MMP-9, is also elevated in arthritis and these enzymes degrade non-collagen matrix components of the joints.

Elevated levels of pro-inflammatory cytokines have been measured in RA and OA synovial fluids, even in the absence of infiltration of neutrophils and macrophages into joint tissues. While the source of the cytokines responsible for the inflammatory effects are not fully elucidated, the fibroblast-like synovial cells, macrophage-like synovial cells, and the chondrocytes, are potential sources.

Inflammation also plays an important role in the bone healing process. TNF-α specifically is one of the initial inflammatory cytokines within the fracture hematoma and initial elevated concentrations are essential for the recruitment of mesenchymal stromal cells (MSCs) into the fracture site. Long-term exposure to elevated inflammatory cytokine levels can be detrimental to bone healing. For example, decreased chondrocyte proliferation and increased apoptosis has been observed in rat metatarsal cultures following extended exposure to IL-1β and TNF-α. Elevated exposure to TNF-α has also been shown to decrease osteoblast calcification and increase osteoclastogenesis, and lead to higher nonunion rates in a murine model. Several studies have shown that the suppressive effects of TNF-α and IL-1β can be alleviated by treatment with IL-1 receptor antagonist (IL-1Ra) and inhibitors for IL-1 or TNF-α.

In the degenerative disc milieu, both TNF-α and IL-1 have been reported to be present at increased levels. The increased levels of pro-inflammatory IL-1β and TNF-α in degenerated discs may drive the environment to a catabolic state. Additionally, in the presence of these inflammatory cytokines, IVD cells produce chemoattractants for macrophages, and significantly downregulated growth differentiation factor 5, an important anabolic factor in the disc. While the majority of the nucleus pulposus portion of the disc is removed during the spinal fusion procedure, this process is not perfect and varies by spine fusion approach. Specifically, it has been found that only 70-80% of the disc was removed during disc preparation using transforaminal lumbar interbody fusion (TLIF). Additionally, the anulus fibrosis is not completely removed during the spine fusion process, suggesting the inflammatory environment of the disc space partially remains. Various cell types play a role in the inflammatory responses driving bone fusion, including MSCs and macrophages.

MSCs have recently been identified as an immunomodulatory cell type, specifically in bone healing. The immunosuppressive activity of MSCs has been shown to be mediated through the NF-κβ pathway, which is activated by TNF-α. Recent studies have demonstrated that exposure to certain toll-like receptors (TLR) polarizes MSCs toward pro- or anti-inflammatory phenotypes. These activated MSCs have been shown to play a role in the reduction of TNF-α and IL-1β after fracture, leading to better regenerative outcomes. Recent studies have also demonstrated the importance of MSC-macrophage crosstalk in bone healing, showing that MSCs suppress the pro-inflammatory responses of macrophages in vitro. Specifically, immune modulation is thought to be one of the primary mechanisms of action for MSC cell therapies, highlighting the role MSCs play in bone healing.

Cytokines and growth factors are produced in joint tissues and released into the synovial fluid, and they act on the resident cells in an autocrine-paracrine manner. While many of these cytokines and growth factors are necessary at low levels for normal homeostasis, when these factors are produced in increased quantities or produced in a temporally unregulated manner they may contribute to the pathogenesis of RA and OA. The major pro-inflammatory cytokines (which are generally catabolic), include, but are not limited to, IL-1α and IL-1β, TNF-α, IL-6, leukemia inhibitory factor, oncostatin-M, IL-8, IL-17, and IL-18. The synoviocytes (the cells in the synovium that line the inner layer of the knee joint) have been shown to produce IL-1β, TNF-α, and MMPs. In addition, synoviocytes can also respond to pro-inflammatory cytokines and other pro-inflammatory signals produced by other inflammatory cells.

Anti-inflammatory cytokines are also produced and may counteract the actions of the pro-inflammatory cytokines. These anti-inflammatory cytokines include, but are not limited to, protease inhibitors (such as matrix metalloprotease 3, TIMP-3), IL-4, IL-10, IL-11, IL-13, IL-1ra, and interferon (IFN)-γ. In addition, members of the transforming growth factor (TGF)-β/bone morphogenetic protein (BMP) family, insulin-like growth factor-I, and fibroblast growth factors (FGFs) are considered to be major anabolic factors for cartilage as they promote synthesis of matrix proteins which may counter the effects of inflammation and cartilage destruction. Further complicating the role of cytokines in the inflammatory process, several of the cytokines may have both pro-inflammatory and anti-inflammatory effects. These cytokines include, but are not limited to, IL-1ra, IL-6, and TGF-β.

Although the pro-inflammatory cytokines and anti-inflammatory cytokines offer promising targets for therapeutic intervention, the art is in need of additional therapeutic treatments that are effective in treating joint disease, including, but not limited to, RA and OA. The use of amnion-derived materials has shown promise in the treatment of joint disease, including, but not limited to, RA and OA, and related diseases and conditions, including through the downregulation of the inflammatory response. However, as amnion-derived materials are derived from human starting material, in many cases the therapeutic effects (for example, the ability to downregulate the inflammatory response) of amnion-derived materials vary from supplier to supplier and even with a product from the same supplier. Furthermore, it is difficult to standardize products containing amnion-derived materials and difficult to determine if a particular disease or condition would be responsive to therapy using a product containing amnion-derived materials.

As a result, there remains a need to a need to control the quality and consistency of amnion-derived material for the treatment of various diseases and conditions. The art is in need of methods to quantify and/or standardize amnion-derived materials and methods to determine if a particular disease or condition would be responsive to therapy using amnion-derived materials. The present disclosure provides a solution to these and other unmet needs.

### SUMMARY OF THE DISCLOSURE

The present disclosure relates to the use of model systems optimized to produce an anti-inflammatory response that models the anti-inflammatory response in human diseases and conditions, such as, but not limited to, joint disease. The disclosed model systems may be used in various methods disclosed herein.

Accordingly, in certain non-limiting embodiments, the present disclosure provides for test methods, compositions, and kits for determining the anti-inflammatory effect of a product, particularly a therapeutic biological product. In certain non-limiting embodiments, the present disclosure provides for test methods, compositions, and kits for determining the effect of a product, particularly a therapeutic biological product, on the production and/or activity of at least one pro-inflammatory cytokine and/or at least one anti-inflammatory cytokine.

In certain non-limiting embodiments, the present disclosure provides for test methods, compositions, and kits for quantitating the anti-inflammatory effect of a product, particularly a therapeutic biological product, by determining the effect of the product on the production and/or activity of at least one pro-inflammatory cytokine and/or at least one anti-inflammatory cytokine.

In certain non-limiting embodiments, the present disclosure provides for test methods, compositions, and kits for standardizing the anti-inflammatory effect of a product, particularly a therapeutic biological product, by determining the effect of the product on the production and/or activity of at least one pro-inflammatory cytokine and/or at least one anti-inflammatory cytokine.

In certain non-limiting embodiments, the present disclosure provides for test methods, compositions, and kits for determining, prior to administration of a product to a subject, whether administration of the product to the subject is likely to produce an anti-inflammatory effect in the subject.

Such test methods, compositions, and kits may be used to determine whether a subject suffering from or likely to suffer from a condition involving inflammation can be successfully treated by a product, particularly a therapeutic biological product.

In certain embodiments, the present disclosure relates to a method for quantitating an anti-inflammatory activity of a product, the method comprising: adding the product or a substance derived from the product to an activated cell or tissue of a test system; incubating the product or the substance derived from the product with the test system for a first period of time; determining the extent the product or the substance derived from the product inhibits or stimulates the production, activity, or both the production and activity of a pro-inflammatory mediator from the activated cell or tissue to generate a product value; and associating the product value with the product. In some embodiments, the method further comprises: determining the extent the product or the substance derived from the product inhibits or stimulates the production, activity, or both the production and activity of an anti-inflammatory mediator to generate an additional product value; and associating the additional product value with the product.

In some embodiments, the present disclosure relates to a method for standardizing an anti-inflammatory activity of a product, the method comprising: adding the product or a substance derived from the product to an activated cell or tissue of a test system; incubating the product or the substance derived from the product with the test system for a first period of time; determining the extent the product or the substance derived from the product inhibits or stimulates the production, activity, or both the production and activity of a pro-inflammatory mediator from the activated cell or tissue to generate a product value; comparing the product value with a reference value to determine a standardized value; and associating the standardized value with the product.

In some embodiments of the method, the reference value is determined by: adding a standard to the activated cell or tissue of the test system; incubating the standard with the test system for the first period of time; and determining the extent the standard inhibits or stimulates the production, activity, or both the production and activity of the pro-inflammatory mediator from the activated cell or tissue to generate the reference value. In some embodiments, the method further comprises the steps of: determining the extent the product or the substance derived from the product inhibits or stimulates the production, activity, or both the production and activity of an anti-inflammatory mediator to generate an additional product value; comparing the additional product value with an additional reference value to determine an additional standardized value; and associating the additional standardized value with the product, wherein the additional standardized value is determined by: adding a standard to the activated cell or tissue of the test system; incubating the standard with the test system for the first period of time; and determining the extent the standard inhibits or stimulates the production, activity, or both the production and activity of the anti-inflammatory mediator from the activated cell or tissue to generate the additional standardized value.

In some embodiments, the present disclosure relates to a method for determining, prior to administration of a product to a subject, whether administration of the product to the subject is likely to produce an anti-inflammatory effect in the subject, the method comprising: adding the product or a substance derived from the product to an activated cell or tissue of a test system; incubating the product or the substance derived from the product with the test system for a first period of time; determining the extent the product or the substance derived from the product inhibits or stimulates the production, activity, or both the production and activity of a pro-inflammatory mediator from the activated cell or tissue to generate a product value; comparing the product value with a reference value to determine a standardized value; and comparing the standardized value to a threshold value, wherein when the standardized value is equal to or greater than the threshold value the anti-inflammatory effect is likely to be produced in the subject.

In some embodiments of the method, the reference value is determined by: adding a standard to the activated cell or tissue of the test system; incubating the standard with the test system for the first period of time; and determining the extent the standard inhibits or stimulates the production, activity, or both the production and activity of the pro-inflammatory mediator from the activated cell or tissue to generate the reference value. In some embodiments, the method further comprises: determining the extent the product or the substance derived from the product inhibits or stimulates the production, activity, or both the production and activity of an anti-inflammatory mediator to generate an additional product value; comparing the additional product value with an additional reference value to determine an additional standardized value, wherein when the additional standardized value is equal to or greater than a threshold value, the anti-inflammatory effect is likely to be produced in the subject. In some embodiments, the additional reference value is determined by: adding a standard to the activated cell or tissue of the test system; incubating the standard with the test system for the first period of time; and determining the extent the standard inhibits or stimulates the production, activity, or both the production and activity of the anti-inflammatory mediator from the activated cell or tissue to generate the additional reference value.

In some embodiments of the method, the product is selected from the group consisting of: an amnion-derived product; and an amnion suspension allograft. In some embodiments of the method, the substance derived from the product is selected from the group consisting of: a conditioned media generated from the product; and a factor isolated from a conditioned media generated from the product.

In some embodiments of the method, the test system is an in vitro system. In some embodiments, the in vitro system comprises a mesenchymal stromal cell line. In some embodiments, the in vitro system comprises a transformed human synovial cell line that retains an activatable phenotype. In some embodiments, the cell line is a SW982 cell line.

In some embodiments, the method further comprises the step of producing the activated cell or tissue by contacting the cell or tissue of the test system with an activating factor for a second period of time. In some embodiments, the second period of time is 24 to 96 hours. In some embodiments, the activating factor is selected from the group consisting of: IL-1β; TNF-α; and a combination of both IL-1β and TNF-α. In some embodiments, the activating factor is IL-1β at a concentration of 0.25 to 6 ng/ml and TNF-α at a concentration of 1 to 15 ng/ml.

In some embodiments of the method, the first period of time is 24 to 72 hours. In some embodiments, the method further comprises the step of a manufacturer or vendor making a decision whether the product is suitable for use based on the product value. In some embodiments, the method further comprises the step of a healthcare provider making a treatment decision based on the product value. In some embodiments, the method further comprises the step of a manufacturer or vendor making a decision whether the product is suitable for use based on the product value, the additional product value, or both the product value and the additional product value. In some embodiments, the method further comprises the step of a healthcare provider making a treatment decision based on the product value, the additional product value, or both the product value and the additional product value.

In some embodiments of the method, the pro-inflammatory mediator is a cytokine. In some embodiments, the cytokine is selected from the group consisting of: IL-1α and IL-1β; TNF-α; IL-6; leukemia inhibitory factor; oncostatin-M; IL-8; IL-17; and IL-18. In some embodiments, the pro-inflammatory mediator is a protease. In some embodiments, the protease is a matrix metalloprotease. In some embodiments, the anti-inflammatory mediator is a cytokine. In some embodiments, the cytokine is selected from the group consisting of: IL-4; IL-6; IL-10; IL-11; IL-13; IL-1 receptor antagonist (IL-1ra); IFN-γ; TGF-β; bone morphogenic protein; insulin-like growth mediator-I; and fibroblast growth mediator. In some embodiments, the anti-inflammatory mediator is a protease inhibitor. In some embodiments, the protease inhibitor is a matrix metalloprotease inhibitor.

### BRIEF DESCRIPTION OF THE DRAWINGS

The following detailed description of the present disclosure may be better understood, by way of example only, with reference to the following drawings.

FIG. 1 is graphical representation of ELISA data displaying effects of higher passage (“old cells”) versus lower passage (“new cells”) cells, cell density, and randomization of well conditions on TNF-α protein levels. Average±standard deviation is reported; n=3 per group. **** denotes p<0.0001 by Dunnett's multiple comparisons test to appropriate INF cell condition control. AM represents assay media; INF represents activating factor alone in AM. High passage refers to cells that were continually passaged (≥10 times) and may have reached confluency or over-confluency in the flask during continual maintenance. The term low passage refers to cells that were thawed and passaged between about 1 and 5 times before use in the assays.

FIG. 2 is graphical representation of ELISA data displaying effects of CO₂ on TNF-α protein levels. Average±standard deviation reported; n=3 per group. **** denotes p<0.0001 by Dunnett's multiple comparisons test relative to the appropriate INF cell condition control. AM represents assay media; INF represents activating factor alone in AM.

FIG. 3 is graphical representation of ELISA data displaying effects of activating factor concentration on TNF-α protein levels. Average±standard deviation reported; n=1-3 per group. ** denotes p<0.01, *** denotes p<0.001, and **** denotes p<0.0001 by Dunnett's multiple comparisons test relative to the appropriate INF cell condition control. AM represents assay media; INF represents activating factor alone in AM.

FIG. 4 is graphical representation of percent reduction of inflammation as indicated by TNF-α protein levels after in the presence or absence of 100% or 50% amnion suspension allograft conditioned media. Average±standard deviation reported; n=1-3 per group. ** denotes p<0.01, *** denotes p<0.001, and **** denotes p<0.0001 by Dunnett's multiple comparisons test relative to the appropriate INF cell condition control. AM represents assay media; INF represents activating factor alone in AM.

FIG. 5 is graphical representation of ELISA data displaying effects of activating factor on TNF-α protein levels after 72 hours of priming with activating factor (immediately before treatment with amnion suspension allograft conditioned media, t0). Average±standard deviation is reported; n=3 per group. *** denotes p<0.001 and **** denotes p<0.0001 by Dunnett's multiple comparison test relative to the appropriate AM cell condition control. AM represents assay media; INF represents activating factor alone in AM.

FIG. 6 is graphical representation of ELISA data displaying TNF-α protein levels in no cell controls. Average±standard deviation reported; n=3 per group for NuCel (an ASA available from Organogenesis, Canton, Mass.)/pooled positive control (PPC) conditioned media (CM); n=1 per group for no cell controls. AM represents assay media; INF represents activating factor alone in AM; P represents PPC.

FIG. 7 is graphical representation of percent reduction in TNF-α protein levels for NuCel CM and PPC CM. Average±standard deviation reported; n=3 per group. INF represents activating factor alone in AM.

FIG. 8 is graphical representation of ELISA data displaying effects of several test articles (TA) TA1, TA2, and PPC CM treatment on TNF-α protein levels. Average±standard deviation is reported; n=3 per group. *** denotes p<0.001, **** denotes p<0.0001 by Dunnett's multiple comparisons test relative to INF control. AM represents assay media; INF represents activating factor alone in AM.

FIG. 9 is graphical representation of ELISA data displaying effects of NuCel CM and PPC CM mini-dose curve treatment on TNF-α protein levels. Average±standard deviation is reported; n=3 per group. ** denotes p<0.01, *** denotes p<0.001 by Dunnett's multiple comparisons test relative to INF control. AM represents assay media; INF represents activating factor alone in AM.

FIG. 10 is graphical representation of ELISA data displaying TNF-α protein levels in no cell controls. Average±standard deviation is reported; n=3 per group for TAI/TA2/PPC CM; n=1 per group for no cell controls. AM represents assay media; INF represents activating factor alone in AM; P represents pooled positive control (PPC).

FIG. 11 is graphical representation of ELISA data displaying TNF-α protein levels in no cell controls. Average±standard deviation is reported; n=3 per group for NuCel/PPC CM; n=1 per group for no cell controls. AM represents assay media; INF represents activating factor alone in AM; P represents pooled positive control (PPC).

FIG. 12 is graphical representation of ELISA data displaying effects of PPC CM treatment on TNF-α protein levels from FIGS. 8 and 9 between two operators. Average±standard deviation is reported; n=3 per group. AM represents assay media; INF represents activating factor alone.

FIG. 13 is graphical representation of ELISA data displaying percent reduction in TNF-α protein levels for PPC CM from FIGS. 8 and 9 between two operators. Average±standard deviation reported; n=3 per group. INF represents inflammation alone in AM.

FIG. 14 is graphical representation of ELISA data displaying TNF-α protein levels for recovery controls. Average reported; n=1 per group. ASA represents amniotic suspension allograft; CM represents conditioned media.

FIG. 15A is a chart showing the effect of ASA treatment on TNF-α in an HFLS model.

FIG. 15B is a chart showing the effect of ASA treatment on IL-1β in an HFLS model. FIG. 15C is a chart showing the effect of ASA treatment on IL-1ra in an HFLS model. * denotes p<0.05 by paired t-test compared to untreated group.

FIG. 16A is a chart showing the effect of ASA CM on TNF-α in a HIG82 model. FIG. 16B is a chart showing the effect of individual component treatment on TNF-α in a HIG82 model. **=p<0.01 ***=p<0.001, One-way analysis of variance (ANOVA) and a Dunnett's multiple comparisons test. Hashed bars indicate 50% CM of ASA. AM=Assay media alone with no inflammatory cytokines or ASA added; CM=ASA-conditioned medium.

FIG. 17A is a chart showing the effect of ASA CM on TNF-α in a SW982 Model. FIG. 17B is a chart showing the effect of individual component treatment on TNF-α in a SW982 Model. **=p<0.01 ***=p<0.001 ****=p<0.0001, One-way analysis of variance (ANOVA) and a Dunnett's multiple comparisons test. Hashed bars indicate 50% CM of ASA. AM=Assay media alone with no inflammatory cytokines or ASA added; CM=ASA-conditioned medium.

FIG. 18 is a chart showing the percent reduction in TNF-α Levels with ASA treatment in an SWA982 Model.

FIG. 19A is a chart showing the effect of ASA treatment on TNF-α protein levels for hTERT-adMSCs. FIG. 19B is a chart showing the effect of ASA treatment on TNF-α protein levels for primary bmMSCs. Average±standard deviation reported; n=3 per group. *** denotes p<0.001, **** denotes p<0.0001 by Tukey's multiple comparisons test within each plate.

FIG. 20A is a chart showing the effect of ASA treatment on TNF-α protein levels for hTERT-adMSCs. FIG. 20B is a chart showing the effect of ASA treatment on TNF-α protein levels for primary bmMSCs. TNF-α levels in no cell controls (hashed) compared to treated cells (solid bars, same as shown in FIG. 19). Average±standard deviation reported; n=3 per group (solid bars), n=1 per group for no cell controls (hashed bars).

FIG. 21A is a chart showing the percent reduction in TNF-α protein levels from inflammation alone for hTERT-adMSCs. FIG. 21B is a chart showing the percent reduction in TNF-α protein levels from inflammation alone for primary bmMSCs. Average±standard deviation reported; n=3 per group.

FIG. 22 are charts showing the effect of ASA treatment on TNF-α protein levels. Average±standard deviation reported; n=3 per group. *** denotes p<0.001, **** denotes p<0.0001 by Tukey's multiple comparisons test within each plate.

FIG. 23 is a chart showing the effect of ASA treatment on TNF-α protein levels. TNF-α levels in no cell controls (hashed) compared to supernatant from treated cells (solid bars, same as shown in FIG. 22). Average±standard deviation reported; n=3 per group (solid bars), n=1 per group (hashed bars).

FIG. 24 is a chart showing the percent reduction in TNF-α protein levels from inflammation alone. Average±standard deviation reported; n=3 per group.

### DETAILED DESCRIPTION

The use of amnion-derived materials has shown promise in the treatment of a variety of diseases and conditions, including, but not limited to, joint diseases. In particular amnion-derived products have been used to treat OA, RA, spinal fusion and bone healing.

Unfortunately, the therapeutic impact of amnion-derived products is difficult to predict, due at least partially to the differing properties between amnion-derived products. Furthermore, amnion-derived products are not typically standardized with respect to a standard value to allow the therapeutic potential of the amnion-derived products to be estimated. As a result, there remains a need to control the quality and consistency of amnion-derived products for the treatment of various diseases and conditions, including, but not limited to, joint disease.

The present disclosure provides methods to quantify and/or standardize an activity, preferably an anti-inflammatory activity, of products, particularly amnion-derived products. As a result, the present disclosure provides a solution to the shortcoming and needs of the art. In addition, the present disclosure provides methods for determining, prior to the administration of a product (particularly an amnion-derived product) to a subject, if the product is likely to produce an anti-inflammatory effect when such product is administered to the subject. Through the use of the methods disclosed, the present disclosure enables the more widespread use of products, including but not limited to amnion-derived products, for a variety of therapeutic applications.

### Test System and Activated Test System

A variety of test systems may be used in the methods, compositions and kits of the present disclosure. The test system may be an in-vitro test system or an in-vivo test system. In a preferred embodiment, the test system is an in vitro test system.

The test system comprises a cell or tissue that is representative of the pathophysiology of a disease state or condition to be tested. The cell or tissue may be from any living organism. Preferably, the cell or tissue is from a mammal, more preferably from a primate, more preferably from a human. For example, for a test system for investigating joint disease, the test system may comprise a cell or tissue representative of the pathophysiology of the joint disease. In one embodiment, the cell is a transformed cell line. In another embodiment, the cell is a non-transformed cell line. In one embodiment, the test system comprises a cell line that is representative of the pathophysiology of OA and the test system is preferably an in-vitro system. In one embodiment, the test system comprises a cell line that is representative of the pathophysiology of OA and the test system is preferably an in-vitro system.

When the test system is used to investigate a joint disease, the test system preferably incorporates a cell or tissue that is capable of producing an inflammatory response. The cell or tissue of the test system preferably retains the “activatable phenotype” observed in primary synoviocyte cultures, meaning the cell or tissue produces an inflammatory response when stimulated with activating factors. The use of a transformed cell line with an activatable phenotype allows study of the inflammatory response typical of synoviocytes without the variation and expansion limit characteristics of primary synoviocytes cultures. Cell lines for use in a test system that is representative of the pathophysiology of joint disease include, but are not limited to, human fibroblast-like synoviocytes (HFLS; human primary cell line from synovial tissue; Cell Applications, Inc. Cat No. 408K-05a), human macrophage-like synoviocyte (HMLS; human primary cell line from synovial tissue), HIG-82 (a rabbit synovial cell line; ATCC Cat. No. CRL-1832; Georgescu et al., In Vitro Cellular & Developmental Biology, 24(10), pages 10151022, 1988), SW982 (a human synovial sarcoma cell line; ATCC Cat. No. HTB-93), mesenchymal stromal cells, including but not limited to primary bone marrow-derived MSCs (bmMSCs) and hTERT immortalized adMSCs (ASC52telo; hTERT-adMSCs).

The cell or tissue of the test system is subject to activation by an activating factor. Representative activating factors include, but are not limited to, phorbol myristate acetate (PMA), IL-1β, and TNF-α. When a cell or tissue that has an activatable phenotype is stimulated with an activating factor, the cell line or tissue responds by producing an inflammatory response, including producing pro-inflammatory mediators, that mimics the inflammatory response observed in human disease. Pro-inflammatory mediators include, but are not limited to, growth factors, cytokines, and proteases. Representative pro-inflammatory growth factors and cytokines include, but are not limited to, IL-1α and IL-1β, TNF-α, IL-6, leukemia inhibitory factor, oncostatin-M, IL-8, IL-17, and/or IL-18. Representative pro-inflammatory proteases include, but are not limited to MMPs, such as, but not limited to, MMP-1, MMP-2, MMP-3, MMP-9, and/or MMP-13. Preferred pro-inflammatory mediators include, but are not limited to, IL-1β, TNF-α, MMP1, and/or MMP-13. When a cell or tissue that has an activatable phenotype is stimulated with an activating factor, the cell line or tissue may also respond by producing an anti-inflammatory response, including producing anti-inflammatory mediators, that mimics the inflammatory response observed in human disease. Anti-inflammatory mediators include, but are not limited to, growth factors, cytokines, and proteases. Representative anti-inflammatory cytokines and growth factors include, but are not limited to, IL-4, IL-6, IL-10, IL-11, IL-13, IL-1 receptor antagonist (IL-1ra), IFN-γ, TGF-β, bone morphogenic protein, insulin-like growth factor-I, and/or fibroblast growth factor. Representative anti-inflammatory protease inhibitors include, but are not limited to, metalloprotease inhibitor 3 (TIMP-3). Preferred anti-inflammatory mediators include, but are not limited to, IL-1ra, IL-6, and/or TIMP-3.

The activating factor may be used at any concentration that induces an inflammatory response. The inflammatory response may be monitored by determining the concentration of one or more pro-inflammatory mediators after the activating factor is added to the test system. In one embodiment, the activating factor is used at a concentration that induces the maximum production (or within 30% of the maximum production) of one or more pro-inflammatory mediators. Such a concentration may be determined using a dose response curve at a time point (for example, 24, 48 or 72 hour or more) after the activating factor is added to the test system. Any of the pro-inflammatory mediators discussed above may be monitored to determine the inflammatory response. In a preferred embodiment, IL-1β, TNF-α, MMP1, and/or MMP-13 is monitored. In a further preferred embodiment, TNF-α is monitored.

The activating factor may be incubated with the test system for a period of time sufficient to induce an inflammatory response. The inflammatory response may be monitored by determining the concentration of one or more pro-inflammatory mediators after the activating factor is added to the test system. A concentration of the activating factor that induces the maximum production (or within 30% of the maximum production) of one or more pro-inflammatory mediators may be used to determine such a period of time. In one embodiment, the activating factor is incubated with the test system for a period of time that induces the maximum production (or within 30% of the maximum production) of one or more pro-inflammatory mediators. Such a time period may be determined by determining the concentration of one or more pro-inflammatory mediators over time (for example, 24, 48 or 72 hour or more) after the activating factor is added to the test system. Any of the pro-inflammatory mediators discussed above may be monitored to determine the inflammatory response. In a preferred embodiment, IL-1β, TNF-α, MMP1, and/or MMP-13 is monitored. In a further preferred embodiment, TNF-α is monitored.

In one embodiment, the activating factor is TNF-α. When TNF-α is the activating factor, TNF-α may be added to or contacted with the test system at any concentration that induces an inflammatory response as discussed above. In one embodiment, TNF-α is used at a concentration ranging from 0.1 to 50 ng/ml. In certain preferred embodiments, the concentration of TNF-α is from 0.1 to 30 ng/ml, 0.5 to 20 ng/ml, 0.75 to 15 ng/ml, 1 to 15 ng/ml, 2 to 10 ng/ml, 5 to 15 ng/ml or 8 to 12 ng/ml. In another preferred embodiment, the concentration of TNF-α is 10 ng/ml.

When TNF-α is the activating factor, TNF-α may be incubated with the test system for a period of time sufficient to generate an inflammatory response as discussed above. In one embodiment, the period of time is from 12 hours to 144 hours. In certain preferred embodiments, the period of time is from 12 to 120 hours, 12 to 96 hours, 12 to 72 hours, 24 to 72 hours, 48 to 72 hours, 60 to 80 hours, or 65 to 75 hours. In another preferred embodiment, the period of time is 72 hours. Any concentration of TNF-α in the preceding paragraph may be used in combination with the disclosed time periods. In a preferred embodiment, the concertation of TNF-α used in combination with the disclosed time periods is 8-12 ng/ml or 10 ng/ml.

In another embodiment, the activating factor is IL-1β. When IL-1β is the activating factor, IL-1β may be added to or contacted with the test system at any concentration that induces an inflammatory response as discussed above. In one embodiment, IL-1β is used at a concentration ranging from 0.01 to 10 ng/ml. In certain preferred embodiments, the concentration of IL-1β is from 0.01 to 8 ng/ml, 0.1 to 7 ng/ml, 0.25 to 6 ng/ml, 0.5 to 5 ng/ml, 0.5 to 4 ng/ml, 0.75 to 3 ng/ml or 0.5 to 2 ng/ml. In another preferred embodiment, the concentration of IL-1β is 1 ng/ml.

When IL-1β is the activating factor, IL-1β may be incubated with the test system for a period of time sufficient to generate an inflammatory response as discussed above. In one embodiment, the period of time is from 12 hours to 144 hours. In certain preferred embodiments, the period of time is from 12 to 120 hours, 12 to 96 hours, 12 to 72 hours, 24 to 72 hours, 48 to 72 hours, 60 to 80 hours, or 65 to 75 hours. In another preferred embodiment, the period of time is 72 hours. Any concentration of IL-1β in the preceding paragraph may be used in combination with the disclosed time periods. In a preferred embodiment, the concertation of IL-1β used in combination with the disclosed time periods is 0.5 to 2 ng/ml or 1 ng/ml.

In another embodiment, the activating factor is IL-1β and TNF-α. When IL-1β and TNF-α are the activating factors, IL-1β and TNF-α may be added to or contacted with the test system at any concentration that induces an inflammatory response as discussed above. In one embodiment, the TNF-α is used at a concentration ranging from 0.1 to 50 ng/ml and IL-1β is used at a concentration ranging from 0.01 to 10 ng/ml. In certain preferred embodiments, the concentration of TNF-α is from 0.1 to 30 ng/ml, 0.5 to 20 ng/ml, 0.75 to 15 ng/ml, 1 to 15 ng/ml, 2 to 10 ng/ml, 5 to 15 ng/ml or 8 to 12 ng/ml and the concentration of IL-1β is from 0.01 to 8 ng/ml, 0.1 to 7 ng/ml, 0.25 to 6 ng/ml, 0.5 to 5 ng/ml, 0.5 to 4 ng/ml, 0.75 to 3 ng/ml or 0.5 to 2 ng/ml. In another preferred embodiment, the concentration of TNF-α is 8-12 ng/ml and the concentration of IL-1β is 0.5 to 2 ng/ml. In another preferred embodiment, the concentration of TNF-α is 10 ng/ml and the concentration of IL-1β is 1 ng/ml.

When IL-1β and TNF-α are the activating factors, IL-1β and TNF-α may be incubated with the test system for a period of time sufficient to generate an inflammatory response as discussed above. In one embodiment, the period of time is from 12 hours to 144 hours. In certain preferred embodiments, the period of time is from 12 to 120 hours, 12 to 96 hours, 12 to 72 hours, 24 to 72 hours, 48 to 72 hours, 60 to 80 hours, or 65 to 75 hours. In another preferred embodiment, the period of time is 72 hours. Any concentration of IL-1β and TNF-α in the preceding paragraphs may be used in combination with the disclosed time periods. In a preferred embodiment, the concentration of TNF-α is 8-12 ng/ml and the concentration of IL-1β is 0.5 to 2 ng/ml. In another preferred embodiment, the concentration of TNF-α is 10 ng/ml and the concentration of IL-1β is 1 ng/ml.

After the activating factor is added to the test system, the cell or tissue of the test system becomes an activated cell or tissue. As the activated cell or tissue of the test system responds to the activating factor with an increase in the production and/or activity of pro-inflammatory mediators and in certain cases an increase in the production and/or activity of anti-inflammatory mediators, the test system allows for determining the effect of a product or a substance derived from the product on the production and/or activity of pro-inflammatory mediators and anti-inflammatory mediators. A product useful for treating joint disease would preferably decrease the production and/or activity of one or more pro-inflammatory mediators, increase the production and/or activity of one or more anti-inflammatory mediators, or decrease the production and/or activity of one or more pro-inflammatory mediators and at the same time increase the production and/or activity of one or more anti-inflammatory mediators. Such pro-inflammatory mediators and anti-inflammatory mediators are described herein. Preferably the product or a substance derived from the product inhibits the production and/or activity of IL-1β, TNF-α, and/or an MMP (such as, but not limited to, MMP-1 and MMP-13) and/or increases the production and/or activity of IL-1ra, IL-6, and/or TIMP-3.

In determining if a product or a substance derived from the product decreases the production and/or activity of one or more pro-inflammatory mediators or increases the production and/or activity of one or more anti-inflammatory mediators, the effect of a product or a substance derived from the product is compared to a control that is exposed to the activating factor but not exposed to the product. The pro-inflammatory and anti-inflammatory mediators may be detected by any means known in the art. When a protein is detected, a preferred method of detection is ELISA. Commercially available ELISA kits are available for most if not all the mediators discussed herein. Antibodies specific for the various proteins may be produced as well and used with commercially available reagents. When a nucleic acid is detected, the preferred method of detection is a PCR assay. Commercially available PCR kits are available for most if not all the mediators discussed herein. Probes and/or primers specific for the various nucleic acids may be produced as well and used with commercially available reagents.

As used herein, the term “production” means either the synthesis of mRNA that encodes a particular mediator and/or the synthesis of a protein corresponding to a particular mediator. In some cases, a decrease in the production of nucleic acid (for example mRNA) occurs at an early time point and a decrease in the production of protein occurs at a later time point (when the production of nucleic acid may or may not be decreased). Therefore, in certain embodiments, the term production refers to the synthesis of nucleic acid and the determination of the effect of the product or a substance derived from the product on nucleic acid synthesis is determined 2 to 48 and/or 48 to 96 hours after the product or a substance derived from the product is added to or contacted with the test system. In certain embodiments, the term production refers to the synthesis of protein and the determination of the effect of the product or a substance derived from the product on protein synthesis is determined 2 to 48 and/or 48 to 96 hours after the product or a substance derived from the product is added to or contacted with the test system. In one preferred embodiment, a decrease in production refers to a decrease in the production of protein. In another preferred embodiment, a decrease in production refers to a decrease in the production of protein particularly when the effect is determined at a later time point (for example, 48 to 96 hours after the product or a substance derived from the product is added to or contacted with the test system).

A decrease in the production of a pro-inflammatory mediator means a decrease in the production of nucleic acid encoding the pro-inflammatory mediator, a decrease in the production of the pro-inflammatory mediator protein, or a combination of the foregoing. In certain embodiments, a decrease in the production of nucleic acid encoding the pro-inflammatory mediator is determined at an early time point (for example, 2-48 hours) and/or a later time point (for example, 48-96 hours) after the product or a substance derived from the product is added to or contacted with the test system. In certain embodiments, a decrease in the production of pro-inflammatory mediator protein is determined at an early time point (for example, 2-48 hours) and/or a later time point (for example, 48-96 hours) after the product or a substance derived from the product is added to or contacted with the test system.

In one preferred embodiment, a decrease in production refers to a decrease in the production of TNF-α protein. In another preferred embodiment, a decrease in production refers to a decrease in the production of TNF-α protein, particularly when the effect is determined at an early time point (for example, 2-48 hours) and/or a later time point (for example, 48-96 hours) after the product or a substance derived from the product is added to or contacted with the test system.

In another preferred embodiment, a decrease in production refers to a decrease in the production of IL-1β protein. In another preferred embodiment, a decrease in production refers to a decrease in the production of IL-1β protein, particularly when the effect is determined at an early time point (for example, 2-48 hours) and/or a later time point (for example, 48-96 hours) after the product or a substance derived from the product is added to or contacted with the test system.

In another preferred embodiment, a decrease in production refers to a decrease in the production of MMP protein. In another preferred embodiment, a decrease in production refers to a decrease in the production of MMP protein, particularly when the effect is determined at an early time point (for example, 2-48 hours) and/or a later time point (for example, 48-96 hours) after the product or a substance derived from the product is added to or contacted with the test system. In certain aspects, the MMP is MMP-1, MMP-13, MMP-2, MMP-3, or MMP-9. In certain aspects, the MMP is MMP-1 or MMP-13.

As another example, an increase in the production of an anti-inflammatory mediator means an increase in the production of nucleic acid (such as mRNA) encoding the anti-inflammatory mediator, an increase in the production of the anti-inflammatory mediator protein, or a combination of the foregoing. In certain embodiments, an increase in the production of RNA encoding the anti-inflammatory mediator is determined at an early time point (for example, 2-48 hours) and/or a later time point (for example, 48-96 hours) after the product or a substance derived from the product is added to or contacted with the test system. In certain embodiments, an increase in the production of anti-inflammatory mediator protein is determined at an early time point (for example, 2-48 hours) and/or a later time point (for example, 48-96 hours) after the product or a substance derived from the product is added to or contacted with the test system.

In one preferred embodiment, an increase in production refers to an increase in the production of IL-6 protein. In another preferred embodiment, an increase in production refers to an increase in the production of IL-6 protein, particularly when the effect is determined at an early time point (for example, 2-48 hours) and/or a later time point (for example, 48-96 hours) after the product or a substance derived from the product is added to or contacted with the test system.

In one preferred embodiment, an increase in production refers to an increase in the production of IL-1ra protein. In another preferred embodiment, an increase in production refers to an increase in the production of IL-1ra protein, particularly when the effect is determined at an early time point (for example, 2-48 hours) and/or a later time point (for example, 48-96 hours) after the product or a substance derived from the product is added to or contacted with the test system.

In one preferred embodiment, an increase in production refers to an increase in the production of TIMP-3 protein. In another preferred embodiment, an increase in production refers to an increase in the production of TIMP-3 protein, particularly when the effect is determined at an early time point (for example, 2-48 hours) and/or a later time point (for example, 48-96 hours) after the product or a substance derived from the product is added to or contacted with the test system.

The cell line or tissue of the test system may be maintained or cultured under conditions such that the viability of the cell or tissue is maintained. Those skilled in the art will be able to determine such conditions without undue experimentation. The cell line or tissue of the test system may be maintained or cultured according to the conditions for the cell line or tissue recommended by the vendor. Typical culture conditions for a cell line include the use of an appropriate complete growth medium that supplies the essential nutrients (amino acids, carbohydrates, vitamins, minerals) supplemented with cytokines, growth factors, hormones, and/or fetal bovine/calf serum. The cell line or tissue may be maintained or cultured in an appropriate controlled environment with the appropriate concentration of O₂ and/or CO₂ and in a regulated physicochemical environment (for example, pH, osmotic pressure, and temperature).

When the cell or tissue is exposed to an activating factor, a product, or a substance derived from a product, assay media may be used. Assay media differs from growth media in that the concentration of supplemented cytokines, growth factors, hormones, and/or fetal bovine/calf serum is less than that of the growth media, for example, 50% less or more. In certain embodiments, assay media contains 0 to 50% of fetal bovine/calf serum and no additional cytokines, growth factors, or hormones. Preferably in the test systems described herein, growth media is used to culture the cells to an appropriate density. Once the cells have reached an appropriate density, growth media is replaced by assay media for exposure of the test system to an activating factor, a product and/or a substance derived from a product.

In a preferred embodiment, the cell line is the SW982 cell line. SW982 cells may be maintained and cultured as described herein. In one embodiment, the SW982 cells are cultured in Leibowitz's L-15 medium (HyClone, Chicago, Ill.) supplemented with 10% fetal bovine serum (Corning, Corning, N.Y.) (L-15 growth medium). SW982 cells were cultured and passaged according to ATCC protocols. As L-15 culture medium is formulated for use in an incubator without CO₂, SW982 cells are cultured in flasks with phenoic-like caps (which prevents air exchange with the incubator) in an incubator without CO₂.

Assay medium for SW982 cells contains reduced concentration cytokines, growth factors, hormones and fetal bovine serum as compared to the L-15 growth medium. In one embodiment, assay media for SW982 cells comprises 50% (v/v) L-15 growth medium and 50% basal (un-supplemented) L-15 medium.

Product or Substance Derived from a Product

A variety of products may be used in conjunction with the methods, compositions, and kits described herein. In one embodiment, the product is a therapeutic biological product. In a particular embodiment, the product is an amnion-derived product.

The amniotic environment is rich in biological components that have the potential to be supportive of tissue repair following trauma. The placental membrane is comprised of multiple layers of distinct tissues with unique functions. The innermost layer of the membrane is referred to as the amnion, which provides a flexible yet strong barrier to contain the amniotic fluid within the amniotic sac. The chorionic layers of the placental membrane, which adhere to the uterine wall during gestation, are smooth and avascular. Additionally, the amniotic and chorionic membranes contain extracellular matrix components, including collagen, proteoglycans, elastin, laminin, and fibronectin, providing a firm scaffold for cells within the tissue. Gene expression analysis suggests that amniotic membranes may promote an anti-inflammatory environment in the surrounding tissue by suppressing IL-1α and IL-1β and inhibiting the activity of MMPs. Additionally, the amnion contains a number of growth factors, such as, but not limited to, insulin-like growth factor I, TGF-β, fibroblast growth factor, platelet-derived growth factor, and vascular endothelial growth factor which are therapeutically useful.

In a particular embodiment, the product is a human amniotic suspension allograft (ASA). In a particular embodiment, human ASA is obtained from donated human placentas processed in accordance with the Food and Drug Administration's Good Tissue Practices and the American Association of Tissue Bank standards. ASA contains particulated human amniotic membrane and amniotic fluid cells. These components are combined and cryogenically preserved. A preferred ASA is ReNu® (Organogenesis, Canton, Mass.). Another preferred ASA is NuCel® (Organogenesis, Canton, Mass.).

In one embodiment, the substance derived from a product is conditioned media (CM). CM may be generated by culturing the product under appropriate conditions for a period of time (for example 1 to 6 days) and collecting the media; the CM may optionally be sterile filtered and stored for future use (for example at 4° C.). The CM contains factors (such as, but not limited to, cytokines, growth factors, proteases, and other factors) secreted by the product and is used to model the release of growth factors and cytokines from the product when administered to a subject. As such, the CM reflects the factors secreted by the product when administered to a subject.

In one embodiment, the substance derived from a product is CM from ASA. Such CM from ASA may be prepared by incubating the ASA in an appropriate culture media for a period of time, for example 1 to 6 days) and collecting the culture media; the ASA CM may optionally be sterile filtered and stored for future use (for example at 4° C.). The ASA may be incubated in the culture media for any desired period of time. In one embodiment, the period of time is 1 to 6 days. The ASA CM may be generated at any desired temperature, preferably at 4° C. In a particular embodiment, the ASA CM is prepared by incubating ASA in growth medium with 5-10% FBS or in growth medium with 0.1-5% FBS. In one embodiment, the ASA CM is generated by incubating ASA in DMEM with FBS (2.5%), 1% L-glutamine, and 1% penicillin/streptomycin/amphotericin on an orbital rocker for 4 days at 4° C. At the time of use, 100% CM is diluted as required (typically in AM). Where cryopreserved ASA are used, prior to the incubation step the cryopreserved ASA is thawed and processed to remove the DMSO component. The pelleted component of ASA is resuspended in appropriate culture media.

In another embodiment, the substance derived from the product is a factor isolated from a CM generated as described above. In a particular embodiment, the CM from which the factor is isolated is ASA CM. The skilled person will be aware of methods to purify desired factors from the CM.

### Methods for Quantitating an Anti-Inflammatory Effect of a Product

The present disclosure also provides methods for quantitating an anti-inflammatory activity of a product. As discussed herein, many therapeutic biological products have differing activities and/or characteristics. For example, an amnion-derived product, such as an ASA, derived from two donors may have different anti-inflammatory activities. As a result, it would be beneficial to provide methods to quantitate an activity, such as an anti-inflammatory activity, for therapeutic biological products, particularly amnion-derived products such as an ASA.

In one embodiment, the present disclosure provides methods for quantitating an anti-inflammatory activity of a product. In a first embodiment, a method for quantitating an anti-inflammatory activity of a product comprises the steps of: (i) adding an activating factor to a cell or tissue of a test system; (ii) incubating the activating factor with the cell or tissue of the test system for a period of time to generate an activated cell or tissue; (iii) adding a product or a substance derived from the product to the test system; (iv) incubating the product or the substance derived from the product with the activated cell or tissue of the test system for a period of time; (v) determining the extent the product or the substance derived from the product inhibits or stimulates the production, activity, or both the production and activity of a pro-inflammatory mediator from the activated cell or tissue to generate a product value; and (vi) optionally associating the product value with the product. Such a method may further comprise the steps of: (vii) determining the extent the product or the substance derived from the product inhibits or stimulates the production, activity, or both the production and activity of an anti-inflammatory mediator from the activated cell or tissue to generate an additional product value; and (viii) optionally associating the additional product value with the product.

In the method of the first embodiment, any test system described herein may be used and any method of activating a cell or tissue in the test system may be used.

In a preferred aspect of the first embodiment, the cell or tissue is a SW982 cell line, the activating factor is IL-1β and TNF-α, wherein TNF-α is used at a concentration ranging from 0.1 to 50 ng/ml and IL-1β is used at a concentration ranging from 0.01 to 10 ng/ml and the IL-1β and TNF-α are incubated with the test system for 12 hours to 144 hours. In additional preferred aspects of the first embodiment, the concentration of TNF-α is from 0.1 to 30 ng/ml, 0.5 to 20 ng/ml, 0.75 to 15 ng/ml, 1 to 15 ng/ml, 2 to 10 ng/ml, 5 to 15 ng/ml or 8 to 12 ng/ml and the concentration of IL-1β is from 0.01 to 8 ng/ml, 0.1 to 7 ng/ml, 0.25 to 6 ng/ml, 0.5 to 5 ng/ml, 0.5 to 4 ng/ml, 0.75 to 3 ng/ml or 0.5 to 2 ng/ml. In additional preferred aspects of the first embodiment, the concentration of TNF-α is 8-12 ng/ml and the concentration of IL-1β is 0.5 to 2 ng/ml or the concentration of TNF-α is 10 ng/ml and the concentration of IL-1β is 1 ng/ml. In additional preferred aspects of the first embodiment the period of time is from 12 to 120 hours, 12 to 96 hours, 12 to 72 hours, 24 to 72 hours, 48 to 72 hours, 60 to 80 hours, 65 to 75 hours or 72 hours. Any concentration of IL-1β and TNF-α described in this paragraph may be used in combination with the disclosed time periods. Preferably, the concentration of TNF-α is 8-12 ng/ml and the concentration of IL-1β is 0.5 to 2 ng/ml or the concentration of TNF-α is 10 ng/ml and the concentration of IL-1β is 1 ng/ml.

In a second embodiment, a method for quantitating an anti-inflammatory activity of a product comprises the steps of: (i) adding a product or a substance derived from the product to an activated cell or tissue of a test system; (ii) incubating the product or the substance derived from the product with the test system for a period of time; (iii) determining the extent the product or the substance derived from the product inhibits or stimulates the production, activity, or both the production and activity of a pro-inflammatory mediator from the activated cell or tissue to generate a product value; and (iv) optionally associating the product value with the product. Such a method may further comprise the steps of: (v) determining the extent the product or the substance derived from the product inhibits or stimulates the production, activity, or both the production and activity of an anti-inflammatory mediator from the activated cell or tissue to generate an additional product value; and (vi) optionally associating the additional product value with the product.

In the method of the second embodiment, any activated test system described herein may be used. In a particular embodiment of the second aspect, the activated test system is produced as described for preferred aspects of the first embodiment.

In preferred aspects of the methods of the first to second embodiments, the product is preferably an amnion-derived product such as, but not limited to, an ASA. Preferred ASAs include, but are not limited to ReNu® and NuCel® (Organogenesis, Canton, Mass.). In preferred aspects of the methods of the first and second embodiments, the product is incubated with the test system for 2 to 5 days or 3 days.

In preferred aspects of the methods of the first and second embodiments, a substance derived from the product is added to the activated cell or tissue of the test system. Preferably, the substance derived from the product is CM derived from an amnion-derived product such as, but not limited to, an ASA. Preferred ASAs include, but are not limited to ReNu® and NuCel® (Organogenesis, Canton, Mass.). The CM may be 100% CM or the CM may be diluted (such as for example, 75% CM, 50% CM, or 25% CM). When CM is diluted, it is preferably diluted in assay media. In preferred aspects of the methods of the first and second embodiments, the ASA CM is incubated with the test system for 2 to 5 days or 3 days.

In preferred aspects of the methods of the first and second embodiments, the product or substance derived from the product decreases the production and/or activity of one or more pro-inflammatory mediators and/or increases the production and/or activity of one or more anti-inflammatory mediators described herein. Pro-inflammatory mediators that are decreased include, but are not limited to, growth factors, cytokines, and proteases. Representative pro-inflammatory growth factors and cytokines that are decreased include, but are not limited to, IL-1α and IL-1β, TNF-α, IL-6, leukemia inhibitory factor, oncostatin-M, IL-8, IL-17, IL-18. Representative pro-inflammatory proteases that are decreased include, but not limited to MMPs, such as, but not limited to, MMP-1, MMP-2, MMP-3, MMP-9, and/or MMP-13. Preferred pro-inflammatory mediators that are decreased include, but are not limited to, IL-1β, TNF-α, MMP1, and/or MMP-13. Representative anti-inflammatory cytokines and growth factors that are increased include, but are not limited to, IL-4, IL-6, IL-10, IL-11, IL-13, IL-1 receptor antagonist (IL-1ra), IFN-γ, TGF-β, bone morphogenic protein, insulin-like growth factor-I, and/or fibroblast growth factor. Representative anti-inflammatory protease inhibitors that are increased include, but are not limited to, metalloprotease inhibitor 3 (TIMP-3). Preferred anti-inflammatory mediators that are increased include, but are not limited to, IL-1ra, IL-6, and/or TIMP-3.

In third embodiment, a method for quantitating an anti-inflammatory activity of a product comprises the steps of: (i) adding IL-1β and TNF-α to a test system comprising a SW982 cell line; (ii) incubating the IL-1β and TNF-α with the SW982 cell line of the test system for a period of time to generate an activated SW982 cell line; (iii) adding ASA CM to the test system; (iv) incubating the ASA CM with the activated SW982 cell line of the test system for 2 to 4 days; (v) determining the extent the ASA CM inhibits or stimulates the production of TNF-α and optionally one or more additional pro-inflammatory mediators from the activated SW982 cell line to generate a product value; and (vi) optionally associating the product value with the product. Such a method may further comprise the steps of: (vii) determining the extent the ASA CM inhibits or stimulates the production, activity, or both the production and activity of an anti-inflammatory mediator from the activated SW982 cell line to generate an additional product value; and (viii) optionally associating the additional product value with the product.

In a fourth embodiment, a method for quantitating an anti-inflammatory activity of a product comprises the steps of: (i) adding ASA CM to an activated SW982 cell line of a test system; (ii) incubating the ASA CM with the activated SW982 cell line of the test system for 2 to 4 days; (iii) determining the extent the ASA CM inhibits or stimulates the production of TNF-α and optionally one or more additional pro-inflammatory mediators from the activated SW982 cell line to generate a product value; and (iv) optionally associating the product value with the product. Such a method may further comprise the steps of: (v) determining the extent the ASA CM inhibits or stimulates the production, activity, or both the production and activity of an anti-inflammatory mediator from the activated SW982 cell line to generate an additional product value; and (vi) optionally associating the additional product value with the product.

In preferred aspects of the methods of the third and fourth embodiments, the ASA is a human ASA. Preferred ASAs include, but are not limited to ReNu® and NuCel® (Organogenesis, Canton, Mass.).

In preferred aspects of the third and fourth embodiments, the ASA CM is undiluted (i.e., 100% ASA CM). in other preferred aspects, the ASA CM is diluted (such as for example, 75% ASA CM, 50% ASA CM, or 25% ASA CM). When ASA CM is diluted, it is preferably diluted in assay media. In preferred aspects of the methods of the third and fourth embodiments, the ASA CM is incubated with the test system for 3 days.

In preferred aspects of the methods of the third and fourth embodiments, the ASA CM decreases the production of TNF-α and optionally one or more additional pro-inflammatory mediators and/or increases the production and/or activity of one or more anti-inflammatory mediators described herein. Pro-inflammatory mediators that are decreased include, but are not limited to, growth factors, cytokines, and proteases. Representative pro-inflammatory growth factors and cytokines that are decreased include, but are not limited to, IL-1α and IL-1β, IL-6, leukemia inhibitory factor, oncostatin-M, IL-8, IL-17, IL-18. Representative pro-inflammatory proteases that are decreased include, but not limited to MMPs, such as, but not limited to, MMP-1, MMP-2, MMP-3, MMP-9, and/or MMP-13. Preferred pro-inflammatory mediators that are decreased in addition to TNF-α, include, but are not limited to, IL-1β, MMP1, and/or MMP-13. Representative anti-inflammatory cytokines and growth factors that are increased include, but are not limited to, IL-4, IL-6, IL-10, IL-11, IL-13, IL-1 receptor antagonist (IL-1ra), IFN-γ, TGF-β, bone morphogenic protein, insulin-like growth factor-I, and/or fibroblast growth factor. Representative anti-inflammatory protease that are increased inhibitors include, but are not limited to, metalloprotease inhibitor 3 (TIMP-3). Preferred anti-inflammatory mediators that are increased include, but are not limited to, IL-1ra, IL-6, and/or TIMP-3.

In preferred aspects of the methods of the third and fourth embodiments, a manufacturer or vendor makes a decision whether the product is suitable for use and/or a healthcare provider makes a treatment decision based on the product value. In preferred aspects of the methods of the third and fourth embodiments, a manufacturer or vendor makes a decision whether the product is suitable for use and/or a healthcare provider makes a treatment decision based on the product value, the additional product value, or both the product value and the additional product value.

As discussed above, the cell or tissue of the test system responds to the activating factor with an increase in the production and/or activity of pro-inflammatory mediators and/or anti-inflammatory mediators. As a result, the test systems of the present disclosure provide a method to determine the effect of the product, or a substance derived from the product, on the production and/or activity of the pro-inflammatory and anti-inflammatory mediators produced by the cell or tissue of the test system. An output of the test systems described herein is a product value and/or an additional product value that reflects the extent to which the pro-inflammatory mediators are inhibited or anti-inflammatory mediators are stimulated.

As an example product value, if a product was found to inhibit the production of TNF-α by 60% (compared to a control value) and found to result in a TNF-α concentration of 50 pg/ml as determined by the test systems disclosed herein, the product value could be either value. The quantitated product value allows an end-user to gauge the potential activity, such as an anti-inflammatory activity, of the product.

The product value determined as described herein may be used in a number of ways. The product value may be used by a healthcare provider to make a treatment decision. In addition, the product value may be used to accept or reject a product and therefore serve as a reproducible quality control function.

The same approach applies to the additional product value.

### Methods for Standardizing an Anti-Inflammatory Activity of a Product

The present disclosure also provides methods for standardizing an anti-inflammatory activity of a product. As discussed herein, many therapeutic biological products have differing activities and/or characteristics. For example, an amnion-derived product such as an ASA derived from two donors may have a different anti-inflammatory activities. As a result, it would be beneficial to provide methods to standardize an activity, such as an anti-inflammatory activity, for therapeutic biological products, particularly amnion-derived products such as an ASA.

In a fifth embodiment, a method for standardizing an anti-inflammatory activity of a product comprises the steps of: (i) adding an activating factor to a cell or tissue of a test system; (ii) incubating the activating factor with the cell or tissue of the test system for a period of time to generate an activated cell or tissue; (iii) adding a product or a substance derived from the product to the test system; (iv) incubating the product or the substance derived from the product with the activated cell or tissue of the test system for a period of time; (v) determining the extent the product or the substance derived from the product inhibits or stimulates the production, activity, or both the production and activity of a pro-inflammatory mediator from the activated cell or tissue to generate a product value; (vi) comparing the product value with a reference value to determine a standardized value; and (vii) optionally associating the standardized value with the product. Such a method may further comprise the steps of: (viii) determining the extent the product or the substance derived from the product inhibits or stimulates the production, activity, or both the production and activity of an anti-inflammatory mediator from the activated cell or tissue to generate an additional product value; (ix) comparing the additional product value with an additional reference value to determine an additional standardized value; and (x) optionally associating the additional standardized value with the product.

In the method of the fifth embodiment, any test system described herein may be used and any method of activating a cell or tissue in the test system may be used.

In a preferred aspect of the fifth embodiment, the cell or tissue is a SW982 cell line, the activating factor is IL-1β and TNF-α, wherein TNF-α is used at a concentration ranging from 0.1 to 50 ng/ml and IL-1β is used at a concentration ranging from 0.01 to 10 ng/ml and the IL-1β and TNF-α are incubated with the test system for 12 hours to 144 hours. In additional preferred aspects of the fifth embodiment, the concentration of TNF-α is from 0.1 to 30 ng/ml, 0.5 to 20 ng/ml, 0.75 to 15 ng/ml, 1 to 15 ng/ml, 2 to 10 ng/ml, 5 to 15 ng/ml or 8 to 12 ng/ml and the concentration of IL-1β is from 0.01 to 8 ng/ml, 0.1 to 7 ng/ml, 0.25 to 6 ng/ml, 0.5 to 5 ng/ml, 0.5 to 4 ng/ml, 0.75 to 3 ng/ml or 0.5 to 2 ng/ml. In additional preferred aspects of the fifth embodiment, the concentration of TNF-α is 8-12 ng/ml and the concentration of IL-1β is 0.5 to 2 ng/ml or the concentration of TNF-α is 10 ng/ml and the concentration of IL-1β is 1 ng/ml. In additional preferred aspects of the fifth embodiment, the period of time is from 12 to 120 hours, 12 to 96 hours, 12 to 72 hours, 24 to 72 hours, 48 to 72 hours, 60 to 80 hours, 65 to 75 hours or 72 hours. Any concentration of IL-1β and TNF-α described in this paragraph may be used in combination with the disclosed time periods. Preferably, the concentration of TNF-α is 8-12 ng/ml and the concentration of IL-1β is 0.5 to 2 ng/ml or the concentration of TNF-α is 10 ng/ml and the concentration of IL-1β is 1 ng/ml.

In a sixth embodiment, a method for standardizing an anti-inflammatory activity of a product comprises the steps of: (i) adding a product or a substance derived from the product to an activated cell or tissue of a test system; (ii) incubating the product or the substance derived from the product with the test system for a period of time; (iii) determining the extent the product or the substance derived from the product inhibits or stimulates the production, activity, or both the production and activity of a pro-inflammatory mediator from the activated cell or tissue to generate a product value; (iv) comparing the product value with a reference value to determine a standardized value; and (v) optionally associating the standardized value with the product. Such a method may further comprise the steps of: (vi) determining the extent the product or the substance derived from the product inhibits or stimulates the production, activity, or both the production and activity of an anti-inflammatory mediator from the activated cell or tissue to generate an additional product value; (vii) comparing the additional product value with an additional reference value to determine an additional standardized value; and (viii) optionally associating the additional standardized value with the product.

In the method of the sixth embodiment, any test system described herein may be used. In a particular embodiment of the second aspect, the test system is activated as described for preferred aspects of the fifth embodiment.

In preferred aspects of the methods of the fifth to sixth embodiments, the product is preferably an amnion-derived product such as, but not limited to, an ASA. Preferred ASAs include, but are not limited to ReNu® and NuCel® (Organogenesis, Canton, Mass.). In preferred aspects of the methods of the fifth to sixth embodiments, the product is incubated with the test system for 2 to 5 days or 3 days.

In preferred aspects of the methods of the fifth to sixth embodiments, a substance derived from the product is added to the activated cell or tissue of the test system. Preferably, the substance derived from the product is CM derived from an amnion-derived product such as, but not limited to, an ASA. Preferred ASAs include, but are not limited to ReNu® and NuCel® (Organogenesis, Canton, Mass.). The CM may be 100% CM or the CM may be diluted (such as for example, 75% CM, 50% CM, or 25% CM). When CM is diluted, it is preferably diluted in assay media. In preferred aspects of the methods of the first to second embodiments, the ASA CM is incubated with the test system for 2 to 5 days or 3 days.

In preferred aspects of the methods of the fifth to sixth embodiments, the product or substance derived from the product decreases the production and/or activity of one or more pro-inflammatory mediators and/or increases the production and/or activity of one or more anti-inflammatory mediators described herein. Pro-inflammatory mediators that are decreased include, but are not limited to, growth factors, cytokines, and proteases. Representative pro-inflammatory growth factors and cytokines that are decreased include, but are not limited to, IL-1α and IL-1β, TNF-α, IL-6, leukemia inhibitory factor, oncostatin-M, IL-8, IL-17, IL-18. Representative pro-inflammatory proteases that are decreased include, but not limited to MMPs, such as, but not limited to, MMP-1, MMP-2, MMP-3, MMP-9, and/or MMP-13. Preferred pro-inflammatory mediators that are decreased include, but are not limited to, IL-1β, TNF-α, MMP1, and/or MMP-13. Representative anti-inflammatory cytokines and growth factors that are increased include, but are not limited to, IL-4, IL-6, IL-10, IL-11, IL-13, IL-1 receptor antagonist (IL-1ra), IFN-γ, TGF-β, bone morphogenic protein, insulin-like growth factor-I, and/or fibroblast growth factor. Representative anti-inflammatory protease inhibitors that are increased include, but are not limited to, metalloprotease inhibitor 3 (TIMP-3). Preferred anti-inflammatory mediators that are increased include, but are not limited to, IL-1ra, IL-6, and/or TIMP-3.

In seventh embodiment, a method for standardizing an anti-inflammatory activity of a product comprises the steps of: (i) adding IL-1β and TNF-α to a test system comprising a SW982 cell line; (ii) incubating the IL-1β and TNF-α with the SW982 cell line of the test system for a period of time to generate an activated SW982 cell line; (iii) adding ASA CM to the test system; incubating the ASA CM with the activated SW982 cell line of the test system for 2 to 5 days; (iv) determining the extent the ASA CM inhibits or stimulates the production of TNF-α and optionally one or more additional pro-inflammatory mediators from the activated SW982 cell line to generate a product value; (vi) comparing the product value with a reference value to determine a standardized value; and (vii) optionally associating the standardized value with the product. Such a method may further comprise the steps of: (viii) determining the extent the ASA CM inhibits or stimulates the production, activity, or both the production and activity of an anti-inflammatory mediator from the activated SW982 cell line to generate an additional product value; (ix) comparing the additional product value with an additional reference value to determine an additional standardized value; and (x) optionally associating the additional standardized value with the product.

In an eighth embodiment, a method for standardizing an anti-inflammatory activity of a product comprises the steps of: (i) adding ASA CM to an activated SW982 cell line of a test system; (ii) incubating the ASA CM with the activated SW982 cell line of the test system for 2 to 5 days; (iii) determining the extent the ASA CM inhibits or stimulates the production of TNF-α and optionally one or more additional pro-inflammatory mediators from the activated SW982 cell line to generate a product value; (iv) comparing the product value with a reference value to determine a standardized value; and (v) optionally associating the standardized value with the product. Such a method may further comprise the steps of: (vi) determining the extent the ASA CM inhibits or stimulates the production, activity, or both the production and activity of an anti-inflammatory mediator from the activated SW982 cell line to generate an additional product value; (vii) comparing the additional product value with an additional reference value to determine an additional standardized value; and (viii) optionally associating the additional standardized value with the product.

In preferred aspects of the methods of the seventh and eighth embodiments, the ASA is a human ASA. Preferred ASAs include, but are not limited to ReNu® and NuCel® (Organogenesis, Canton, Mass.).

In preferred aspects of the seventh and eighth embodiments, the ASA CM is undiluted (i.e., 100% ASA CM). In other preferred aspects, the ASA CM is diluted (such as for example, 75% ASA CM, 50% ASA CM, or 25% ASA CM). When ASA CM is diluted, it is preferably diluted in assay media. In preferred aspects of the third and fourth embodiments, the ASA CM is incubated with the test system for 3 days.

In preferred aspects of the methods of the seventh and eighth embodiments, the ASA CM decreases the production of TNF-α and optionally one or more additional pro-inflammatory mediators and/or increases the production and/or activity of one or more anti-inflammatory mediators described herein. Pro-inflammatory mediators that are decreased include, but are not limited to, growth factors, cytokines, and proteases. Representative pro-inflammatory growth factors and cytokines that are decreased include, but are not limited to, IL-1α and IL-1β, IL-6, leukemia inhibitory factor, oncostatin-M, IL-8, IL-17, IL-18. Representative pro-inflammatory proteases that are decreased include, but not limited to MMPs, such as, but not limited to, MMP-1, MMP-2, MMP-3, MMP-9, and/or MMP-13. Preferred pro-inflammatory mediators that are decreased in addition to TNF-α, include, but are not limited to, IL-1β, MMP1, and/or MMP-13. Representative anti-inflammatory cytokines and growth factors that are increased include, but are not limited to, IL-4, IL-6, IL-10, IL-11, IL-13, IL-1 receptor antagonist (IL-1ra), IFN-γ, TGF-β, bone morphogenic protein, insulin-like growth factor-I, and/or fibroblast growth factor. Representative anti-inflammatory protease inhibitors that are increased include, but are not limited to, metalloprotease inhibitor 3 (TIMP-3). Preferred anti-inflammatory mediators that are increased include, but are not limited to, IL-1ra, IL-6, and/or TIMP-3.

As discussed above, the cell or tissue of the test system responds to the activating factor with an increase in the production and/or activity of pro-inflammatory mediators and/or anti-inflammatory mediators. As a result, the test systems of the present disclosure provide a method to determine the effect of the product, or a substance derived from the product, on the production and/or activity of the pro-inflammatory and anti-inflammatory mediators produced by the cell or tissue of the test system. An output of the test systems described herein is a product value and/or an additional product value that reflects the extent to which the pro-inflammatory mediators are inhibited or anti-inflammatory mediators are stimulated. The product value and or the additional product value can be compared to a reference value to produce a standardized value and an additional standardized value. In a preferred embodiment, the product value is divided by the reference value. Uses for these values are described herein.

The reference value can be generated in a number of ways. As one example, the reference value can be a pre-determined value. The predetermined value may be determined or set based on information known in the art (for example, a 75% decrease in TNF-α production is known to be beneficial in treating a disease or condition). If a product value for a product is determined to be an 70% decrease in TNF-α production, the standardized value for the product would be 0.93 (the product value divided by the reference value; 0.70/0.75).

Alternatively, the reference value may be determined empirically using a standard. When the reference value is empirically determined, the reference value is preferably determined using the same test system and the same conditions as used to determine the product value. The reference value may be determined at single time and used as the reference value for multiple product values or may be determined separately for each product value (for example, the reference value is determined in parallel with the product value). If the product value for the standard is determined to be a 45 pg/ml TNF-α using the test systems disclosed herein, 45 pg/ml TNF-α is determined to be the reference value. If a product value is determined to be an 50 pg/ml TNF-α, the standardized value for the product would be 1.1 (the product value divided by the reference value; 50/45). A reference value may be determined for a single pro-inflammatory mediator and/or a single anti-inflammatory mediator. A reference value may be determined for multiple pro-inflammatory mediators and/or a single anti-inflammatory mediator. A reference value may be determined for a single pro-inflammatory mediator and/or multiple anti-inflammatory mediators. Further, a reference value may be determined for a group of pro-inflammatory mediators and/or a group of anti-inflammatory mediators.

A variety of standards may be used to generate the reference value. Preferably, the standard is the same as the product. For example, if the product is an ASA, the standard is also an ASA. When the standard and the product are of the same type, the standard may be obtained by pooling various individual products so the standard is reflective of an average activity of the product. In a particular embodiment, the product is an ASA, CM generated from the ASA is added to the test systems described herein, and the standard is pooled CM generated from multiple ASAs (for example, from 5-100 individual ASAs). In another embodiment, the ASA CM and pooled ASA CM are used at 100% (undiluted). In another embodiment, the ASA CM and pooled ASA CM are used at less than 100% (diluted, preferably with appropriate AM), such as, but not limited to 25%, 50, and/or 75%. In another embodiment, the ASA CM and pooled ASA CM are used at 100% (undiluted) and at less than 100% (diluted, preferably with appropriate AM), such as, but not limited to 25%, 50, and/or 75%. In such an embodiment, one or more or all of the pooled ASA CM values may be used as the reference values and compared to the corresponding ASA CM values to generate standardized values as described herein. Such standardized values may be used individually or an average may be taken of one or more or all of the standardized values determined.

Alternatively, the standard may different from the product, such as a small molecule standard to for use with a product which is an ASA. Such a standard may be a concentration of a drug or compound that is known to have a beneficial effect on a disease or condition. Alternatively, the standard may be a concentration of a drug or compound that is known to decrease an inflammatory response in a disease or condition. Further, the standard may be a concentration of a drug or compound that is known to decrease the production and/or activity of a pro-inflammatory mediator, such as, but not limited to TNF-α, in a disease or condition.

The standardized value determined as described herein may be used in a number of ways. The standardized value may be used by a healthcare provider to make a treatment decision. In addition, the standardized value may be used to accept or reject a product and therefore serve as a reproducible quality control function.

The same approach applies to anti-inflammatory mediators, the additional product value, the additional reference value, and the additional standardized value.

### Methods for Determining Whether an Anti-Inflammatory Effect is Likely to be Produced in a Subject by a Product

The present disclosure also provides methods for determining whether an anti-inflammatory effect is likely to be produced in a subject by a product. As many therapeutic biological products have differing activities and/or characteristics, it is difficult to estimate the effect a product might have on a given disease or condition. As inhibiting inflammation is important in treating joint disease, it would be beneficial to provide a method that would inform a user if the product would have a desired anti-inflammatory effect when administered to a subject. For example, a first ASA when administered to a patient completely alleviated the symptoms of OA, while a second ASA when administered to a patient only partially alleviated the symptoms of OA. Such a difference in outcome may be due to the severity of OA between the patients, to a difference in anti-inflammatory activity of the two ASAs, or to the manner in which the two ASAs were administered to the patient. Through the methods described herein, the healthcare provider is able to eliminate the second potential reason for the difference in treatment outcomes.

In a particular embodiment, a subject of the ninth embodiment is suffering from a joint disease. As used herein the term joint disease means those diseases and conditions affecting the joints that involve an upregulated inflammatory response. Representative joint diseases include, but are not limited to, OA, RA, and tendonitis. In another particular embodiment, a subject of the ninth embodiment is suffering from OA. In another particular embodiment, a subject of the ninth embodiment is suffering from RA. In another particular embodiment, a subject of the ninth embodiment is suffering from tendonitis.

In a ninth embodiment, a method for determining whether an anti-inflammatory effect is likely to be produced in a subject by a product prior to administration of the product to the subject is provided. In one aspect of the ninth embodiment, a standard value is determined to according to the methods of any one of the fifth to eighth embodiments described above, including as further described in the preferred aspects applicable to the fifth to eight embodiments. Except as modified below. In addition, this aspect of the ninth embodiment further comprises comparing the standardized value to a threshold value. When the standardized value is equal to or greater than the threshold value, an anti-inflammatory effect is likely to be produced in the subject. When the standardized value is less than the threshold value, an anti-inflammatory effect is not likely to be produced in the subject.

An exemplary method of the ninth embodiment is provided below, where the standardized value is determined according to the eighth embodiment described above.

A method for determining whether an anti-inflammatory effect is likely to be produced in a subject by a product prior to administration of the product to the subject, the method comprising the steps of: (i) adding ASA CM to an activated SW982 cell line of a test system; (ii) incubating the ASA CM with the activated SW982 cell line of the test system for 2 to 5 days; (iii) determining the extent the ASA CM inhibits or stimulates the production of TNF-α and optionally one or more additional pro-inflammatory mediators from the activated SW982 cell line to generate a product value; (iv) comparing the product value with a reference value to determine a standardized value; (v) comparing the standardized value to a threshold value; and (vi) optionally associating the comparison of the standardized value to the threshold value with the product, wherein, when the standardized value is equal to or greater than the threshold value, an anti-inflammatory effect is likely to be produced in the subject.

Such a method may further comprise the steps of: (vii) determining the extent the ASA CM inhibits or stimulates the production, activity, or both the production and activity of an anti-inflammatory mediator from the activated SW982 cell line to generate an additional product value; (viii) comparing the additional product value with an additional reference value to determine an additional standardized value; (ix) comparing the additional standardized value to an additional threshold value; and (vi) optionally associating the comparison of the additional standardized value to the additional threshold value with the product, wherein, when the additional standardized value is equal to or greater than the additional threshold value, an anti-inflammatory effect is likely to be produced in the subject.

As discussed above, the cell or tissue of the test system responds to the activating factor with an increase in the production and/or activity of pro-inflammatory mediators. As a result the test systems of the present disclosure provide a method to determine the likely effect of a product on a disease or condition involving an inflammatory response, such as for example joint disease, by determining the effect of the product, or a substance derived from the product, on the production and/or activity of the pro-inflammatory and/or anti-inflammatory mediators produced by the cell or tissue of the test system. An output of the test systems described herein is a product value and/or an additional product value that reflects the extent to which the pro-inflammatory mediators are inhibited or anti-inflammatory mediators are stimulated. The product value and/or the additional product value can be compared to a reference value or additional reference value to produce a standardized value and an additional standardized value. In a preferred embodiment, the product value/additional product value is divided by the reference value/additional reference value, respectively. Uses for these values are described herein.

The reference value can be generated in a number of ways. As one example, the reference value can be a pre-determined value. The predetermined value may be determined or set based on information known in the art (for example, a 75% decrease in TNF-α production). If a product value for a product is determined to be an 80% decrease in TNF-α production, the standardized value for the product would be 1.1 (the product value divided by the reference value; 0.8/0.75).

Alternatively, the reference value may be determined empirically using a standard. When the reference value is empirically determined, the reference value is preferably determined using the same test system and the same conditions as used to determine the product value. The reference value may be determined at a single time and used as the reference value for multiple product values or may be determined separately for each product value (for example, the reference value is determined in parallel with the product value). If the product value for the standard is determined to be a 60 pg/ml TNF-α using the test systems disclosed herein, 60 pg/ml TNF-α is determined to be the reference value. If a product value is determined to have a 55 pg/ml TNF-α production, the standardized value for the product would be 0.92 (the product value divided by the reference value; 55/60).

A reference value may be determined for a single pro-inflammatory mediator and/or a single anti-inflammatory mediator. A reference value may be determined for multiple pro-inflammatory mediators and/or a single anti-inflammatory mediator. A reference value may be determined for a single pro-inflammatory mediator and/or multiple anti-inflammatory mediators. Further, a reference value may be determined for a group of pro-inflammatory mediators and/or a group of anti-inflammatory mediators.

A variety of standards may be used to generate the reference value. The standard may different from the product, such as a small molecule standard to use with an ASA product. Such a standard may be a concentration of a drug or compound that is known to have a beneficial effect on a disease or condition. Alternatively, the standard may be a concentration of a drug or compound that is known to decrease an inflammatory response in a disease or condition. Further, the standard may be a concentration of a drug or compound that is known to decrease the production and/or activity of a pro-inflammatory mediator, such as, but not limited to TNF-α, in a disease or condition.

Alternatively, the standard is the same as the product. For example, if the product is an ASA, the standard is also an ASA. Preferably, the standard has been shown to have a beneficial effect on a disease or condition and/or been shown to decrease an inflammatory effect in a disease or condition. When the standard and the product are of the same type, the standard may be obtained by pooling various individual products so the standard is reflective of an average activity of the product. In a particular embodiment, the product is an ASA, CM generated from the ASA is added to the test systems described herein, and the standard is pooled CM generated from multiple ASAs (for example, from 5-100 individual ASAs). In another embodiment, the ASA CM and pooled ASA CM are used at 100% (undiluted). In another embodiment, the ASA CM and pooled ASA CM are used at less than 100% (diluted, preferably with appropriate AM), such as, but not limited to 25%, 50, and/or 75%. In another embodiment, the ASA CM and pooled ASA CM are used at 100% (undiluted) and at less than 100% (diluted, preferably with appropriate AM), such as, but not limited to 25%, 50, and/or 75%. In such an embodiment, one or more or all of the pooled ASA CM values may be used as the reference values and compared to the corresponding ASA CM values to generate standardized values as described herein. Such standardized values may be used individually or an average may be taken of one or more or all of the standardized values determined.

In one embodiment, the standardized value determined as described herein is compared to a threshold value to determine if an anti-inflammatory effect is likely to be produced in a subject by administration of a product to the subject. If the standardized value is greater than or equal to the threshold value, the product is determined likely to have an anti-inflammatory effect when administered to a subject. Conversely, if the standardized value is less than the threshold value, the product is determined not likely to have an anti-inflammatory effect when administered to a subject.

The threshold value may be determined based on information known in the art, for example, the knowledge that a 70% decrease in TNF-α production or a TNF-α concentration of less than 50 pg/ml is known to be beneficial in the treatment of a disease or condition or reduce inflammation in a disease or condition. In this approach, only a product with a standardized value of a 70% or greater decrease in TNF-α production or a TNF-α concentration of less than 50 pg/ml is determined likely to have an anti-inflammatory effect when administered to a subject.

The threshold value may also be determined based on the relationship of the product value to the reference value (i.e., the standardized value). The threshold value may be 1.0, such that only a product having a product value greater than or equal to the reference value is deemed likely to have an anti-inflammatory effect when administered to a subject. For example, if the reference value is 60% inhibition of TNF-α production and the product value in 58% inhibition of TNF-α production, the standardized value is 0.97 (the product value divided by the reference value; 0.58/0.60) and the product is determined not likely to have an anti-inflammatory effect when administered to a subject. Alternatively, the threshold value may be 0.75, such that a product having a product value slightly less than or greater than the reference value is deemed likely to have an anti-inflammatory effect when administered to a subject. For example, if the reference value is 80 pg/ml TNF-α and the product value in 60 pg/ml TNF-α, the standardized value is 0.75 (the product value divided by the reference value; 60/80) and the product is determined likely to have an anti-inflammatory effect when administered to a subject. Still further, the threshold value may be 1.1, such that a product having a product value slightly higher than the reference value is deemed likely to have an anti-inflammatory effect when administered to a subject. For example, if the reference value is 59 pg/ml TNF-α and the product value in 71 pg/ml TNF-α, the standardized value is 1.2 (the product value divided by the reference value; 71/59) and the product is determined likely to have an anti-inflammatory effect when administered to a subject.

The threshold value may vary depending on a given disease or condition. For example, the threshold value may be different for OA than for RA. In a preferred embodiment, the threshold value is greater than or equal to the reference value multiplied by 0.75 to 0.99 or 0.85 to 0.99 or 0.95 to 0.99. In another preferred embodiment, the threshold value is greater than or equal to the reference value multiplied by 1 to 1.25 or 1 to 1.15 or 1 to 1.05.

In another embodiment, the standardized value is used directly to determine if an anti-inflammatory effect is likely to be produced in a subject by administration of a product to the subject. In one embodiment, when the standardized value is greater than 0.75, greater than 0.80, greater than 0.85, greater than 0.95 the product is determined likely to have an anti-inflammatory effect when administered to a subject. In another embodiment, when the standardized value is greater than 1, 1.1, 1.2, or 1.25 the product is determined likely to have an anti-inflammatory effect when administered to a subject.

The same approach applies to anti-inflammatory mediators, the additional product value, the additional reference value, and the additional standardized value.

### Kits

The present disclosure also provides for kits for carrying out the methods disclosed, such as the methods of the first through ninth embodiments described herein. In one embodiment, the present disclosure provides a kit for quantitating an anti-inflammatory activity of a product. In another embodiment, the present disclosure provides a kit for standardizing an anti-inflammatory effect of a product. In still another embodiment, the present disclosure provides a kit for determining, prior to administration of a product to a subject, whether administration of the product to the subject is likely to produce an anti-inflammatory effect in the subject.

In certain embodiments, the kits of the present disclosure comprise the required materials and reagents for determining the extent a product or a substance derived from the product inhibits or stimulates the production and/or activity of a pro-inflammatory mediator and/or an anti-inflammatory mediator from an activated cell or tissue described herein.

In certain embodiments, the kits of the present disclosure comprise one or more of the following:

(i) a cell or tissue for use in the methods of the first through ninth embodiment; preferably, the cell or tissue is a human primary cell line or a human transformed cell line; more preferably, the cell or tissue is a human fibroblast-like synoviocyte cell line, a human macrophage-like synoviocyte, a HIG-82 cell line, or a SW982 cell line;

(ii) reagents to stimulate the cell or tissue, particularly the cell lines described in (i), to produce one or more pro-inflammatory mediators and/or one or more anti-inflammatory mediators; preferably, such reagents are IL-1β and/or TNF-α;

(iii) a product, preferably an amnion-derived product, more preferably an ASA product; preferred ASA products are ReNu® or NuCel® (Organogenesis, Canton, Mass.);

(iv) a substance derived from a product, preferably a CM generated by the product, more preferably a CM generated by an ASA described in (iii);

(v) a standard for use in determining a reference value or an additional reference value;

(vi) cell culture media for culturing the cell or tissue of (i) and/or producing the CM of (iv); and

(vii) instructions for carrying out the methods of one or more of the first through ninth embodiments described herein.

### Products and Compositions

The present disclosure also provides for a product or a composition comprising a product, wherein the product or composition has an associated product value. The product value may be directly associated with the product or composition (for example, micro-printed on the product or printed on a container containing the composition) or indirectly associated with the product or composition (for example, printed in materials provided with the product or composition).

The present disclosure also provides for a product or a composition comprising a product, wherein the product or composition has an associated standardized value. The standardized value may be directly associated with the product or composition (for example, micro-printed on the product or printed on a container containing the composition) or indirectly associated with the product or composition (for example, printed in materials provided with the product or composition).

The present disclosure also provides for a product or a composition comprising a product, wherein the product or composition has an associated comparison of a standardized value to a threshold value. The comparison of a standardized value to a threshold value may be directly associated with the product or composition (for example, micro-printed on the product or printed on a container containing the composition) or indirectly associated with the product or composition (for example, printed in materials provided with the product or composition).

In any of the foregoing, when the product is part of a composition, the composition may further comprise a suitable diluent, carrier, and or culture medium.

In any of the foregoing, the product or the composition comprising the product may be cryopreserved.

In any of the foregoing, the product is a biological therapeutic product. In any of the foregoing, the product is an amnion-derived product. In any of the foregoing, the product is a human ASA. In any of the foregoing, the product is ReNu® (Organogenesis, Canton, Mass.). In any of the foregoing, the product is NuCel® (Organogenesis, Canton, Mass.).

### Materials and Methods

In some embodiments, human synovial sarcoma cells (SW982) were obtained from ATCC (Manassas, Va.) and were cultured in Leibowitz's L-15 medium (HyClone, Chicago, Ill.) supplemented with 10% fetal bovine serum (FBS) (Corning, Corning, N.Y.). The SW982 cells are a synovial sarcoma cell line that retains the activatable phenotype of primary synoviocyte cultures without the variation and expansion limits characteristic of primary human cells. However, other cells, such as HIG-82, human fibroblast-like synoviocytes or human macrophage-like synoviocytes are compatible with the disclosed methods. SW982 cells were cultured and passaged according to ATCC protocols. Leibowitz's L-15 culture medium is formulated for use in an incubator without CO₂, and thus cells were cultured in flasks with phenoic-like caps that allow no air exchange with the incubator and placed in an incubator without CO₂. Complete growth media was L-15 media+10% fetal bovine serum. Assay media was 50% (v/v) complete growth medium in L-15 basal media in all experiments.

Conditioned media (CM) was prepared by thawing amniotic suspension allograft (ASA, ReNu® or NuCel®, Organogenesis), followed by centrifugation at 250×g for 5 minutes to remove the DMSO component. The supernatant was removed and discarded and the pelleted component of ASA was then resuspended in assay media at a concentration of 1 mL of assay media per 1 mL of ASA (original volume) and incubated at 4° C. for 4 days with gentle rotation. The suspension was then centrifuged at 250×g for 5 minutes, and then the CM supernatant was collected and sterile filtered using a 0.22 pm polyethersulfone (PES) filter (Millex™-GP Sterile Syringe Filters, SLGP033RS, MilliporeSigma, Burlington, Mass.) before use.

Cell seeding densities range from less than about 25,000 cells/well to more than about 37,000 cells/well, with 25,000 cells/well resulting in appropriate confluency during the course of the disclosed methods. However, other seeding densities are compatible with the disclosed methods and systems. Seeding is undertaken using compatible growth media, which varies with the cell line. Incubation in a CO₂-free incubator results in greater absolute differences in TNF-α production levels between activating factor-only treated cells and ASA CM treated cells, and is thus the preferred incubation condition using SW982 cells. However, CO₂ levels higher than 0%, and specifically 5% or, in some instances >5% CO₂, are possible, especially when other cell lines are utilized. Incubation is generally done at approximately 37° C. The use of Parafilm M™ (Bemis Company, Inc., Neenah, Wis.) or similar self-sealing thermoplastic covering materials is compatible with the disclosed methods, though is optional. Other non-sealable covers are compatible to reduce contamination of cells, though may not prevent or reduce CO₂ access to cells. Cells are seeded in flasks, well plates, or other sterile cell culture containers in various embodiments. These containers are sealed or covered in some instances, or left unsealed or uncovered in other instances. Containers for cell culture are sterilized by ultraviolet irradiation, contact with 70% isopropyl alcohol, or other sterilization means known in the art.

Following overnight attachment, cells are optionally scored or visually evaluated for confluency. However, attachment may occur for longer or shorter periods of incubation time, depending on cell line and growth conditions. Cells are then primed for 72 hours with AM alone, or AM with activating factor included. The priming period is, in some instances, greater or less than 72 hours. Assay media is 50% (v/v) complete L-15 growth medium in L-15 basal media. However, AM may be varied based on the growth medium used, with different percentages of growth medium used in some instances. In certain embodiments, activating factor comprises 1 ng/mL of IL-1β (for example, Invitrogen catalog number RIL1BI, Carlsbad, Calif.) and 10 ng/mL of TNF-α (for example, Millipore catalog number GF023, Burlington, Mass.), however, other concentrations of IL-1β and TNF-α may be used and IL-1β and TNF-α may be used alone at the above concentrations or other concentrations. When an inflammatory response is desired to be greater or less, different concentrations of one or both cytokines are possible. Additionally, other cytokines, such as IL-1α, IL-6, leukemia inhibitory factor, oncostatin-M, IL-8, IL-17, and/or IL-18 are compatible in other embodiments of the disclosed method and may be used in combination with or in the place of IL-1β and/or TNF-α. Activating factor is independently added to AM immediately before use; cytokines are added in small volumes from stock solutions. After 72 hours, samples of the AM and activating factor groups are optionally scored or visually inspected for confluency and all media is exchanged for fresh AM containing fresh activating factor with or with a product or substance derived from the product (for example, ASA CM) to be tested.

When the activating factor is IL-1β and TNF-α, a dose of 10 ng/mL TNF-α provides the largest assay window between assay media and activating factor alone groups when 1 ng/mL IL-1β is used. However, other concentrations of TNF-α and IL-1β are contemplated in the disclosed methods.

For the experiments described in the Examples, cells receive either AM alone, AM with activating factor (such as IL-1β and TNF-α), or AM with activating factor in 100% or 25-75% ASA CM or pooled positive control (PPC) CM. When PPC CM is desired, it is created using NuCel commercial lots, ensuring that PPC CM is generated with roughly equal contributions from various batches of product. The activating factor may be used as described above in any combination previously stated. Inflammatory cytokines are independently added to both AM and CM immediately before use. Additionally, remaining media of approximately 400 μL from each treatment condition is optionally placed into wells or containers without cells. After approximately 48 hours following treatment, a sample from each of the AM and activating factor groups is optionally scored or visually inspected for confluency. The levels of TNF-α in the no cell controls, which consist of remaining media left from each treatment group placed in empty wells or containers and are subject to the same conditions as the cells, are determined when no-cell controls are desired.

Supernatant is collected after approximately 48 hours in culture for analysis of pro-inflammatory mediator/anti-inflammatory mediator production, with TNF-α protein production being a preferred pro-inflammatory factor. However, other time periods of culture prior to analysis are possible. Supernatant is placed at −80° C. and stored until use in enzyme-linked immunosorbent assays (ELISAs). Levels of TNF-α are measured using commercially available kits (for example, DTAOOD, R&D Systems, Minneapolis, Minn.), and ELISAs were performed according to the manufacturer's instructions. For gene expression analysis, cells are collected in 400 μL of RNAzol and stored at −80° C. The commercially available PicoGreen™ assay (Thermo Fisher Scientific, Waltham, Mass.) may be used with the methods disclosed herein.

Additionally, controls such as Quantikine Immunoassay Control Group 248 (QC248, R&D Systems, Minneapolis, Minn.) are optionally utilized. These controls are resuspended and run according to the manufacturer's instructions. A recovery and selectivity control are optionally included to confirm that the TNF-α ELISA is capable of recovering TNF-α protein in the CM. Standard TNF-α is spiked into 50% CM at 500 pg/mL both alone (recovery control) and with 500 pg/mL IL-1β used for priming (selectivity control). However, other amounts of standard TNF-α for assay controls are compatible with the disclosed methods.

When gene expression analysis is desired, cells may be collected as described above. In one embodiment, Direct-zol™ MicroPrep Plus columns (Zymo Research) are used to extract and isolate RNA, and cDNA is obtained via reverse transcription using the Verso cDNA synthesis kit (ThermoFisher Scientific) on a Veriti Thermal Cycler (Applied Biosystems). TaqMan probes (Life Technologies) were used to evaluate the gene expression of relevant targets using a QuantStudio™ 3 Real-Time PCR System (Applied Biosystems). Ct values are obtained from the data using the QuantStudio™ Design and Analysis software, and the 2^{ΔΔCt} method was used to determine fold change compared to a housekeeping gene (glyceraldehyde 3-phosphate dehydrogenase, GAPDH). Suitable TaqMan probes for representative pro-inflammatory and anti-inflammatory mediators are shown in Table 1 below.

TABLE 1

| | Gen | |
|---|---|---|
| Cytokine | Abbreviation | Accession No. |
| glyceraldehyde 3-phosphate | GAPDH | Hs02758991_gl |
| dehydrogenase | | |
| Interleukin-1 beta | IL-1β | Hs00174097 ml |
| Interleukin-1 Receptor antagonist | IL-1ra | Hs00893626 ml |
| Interleukin-6 | IL-6 | Hs00174131ml |
| Matrix Metalloproteinase 1 | MMP-1 | Hs00899658 ml |
| Tissue Inhibitor of | TIMP-3 | Hs00165949 ml |
| Metalloproteinase 3 | | |
| Tumor Necrosis Factor Alpha | TNF-α | Hs01113624_gl |

All statistical analysis is undertaken using commercially available software, such as GraphPad Prism (GraphPad Software). A one-way analysis of variance (ANOVA) and a Dunnett's multiple comparisons test are run. For all graphs, average±standard deviation is reported. * denotes p<0.05, ** denotes p<0.01, ***denotes p<0.001, and **** denotes p<0.0001. For all graphs, the solid bars represent 100% dose and the hashed bars represent 50% dose. Other forms of statistical analysis are performed in embodiments not described herein and are compatible with the disclosed methods.

### EXAMPLES

The following examples illustrate certain aspects of the above-described methods and results. The following examples are shown by way of illustration only and not by way of limitation.

### Example 1

The effects of high passage (old cells) and low passage (new cells) at cell densities of 37,000 cells/well and 25,000 cells/well were examined. In this example, the term high passage refers to cells that have been continually passaged (>10 times) and may have reached confluency or over-confluency in the flask during continual maintenance. The term low passage refers to cells that have been thawed and passaged between about 1 and 5 times before use in the assays, and these cells were carefully managed during passaging to avoid cells reaching >90% confluency between passages.

SW982 were seeded at a density of 25,000 cells/well or 37,000 cells/well in 800 μL of growth media in a 12 well plate and allowed to attach overnight, with 7 plates prepared in total. Four plates had SW982 cells at passage 30 (“new cells”) at 25,000 cells/well, one plate had SW982 cells at passage 30 (“new cells”) at 37,000 cells/well, one plate had SW982 cells at passage 37 at 37,000 cells/well (“old cells”), and one plate had SW982 cells at passage 37 at 25,000 cells/well (“old cells”). Growth media was Leibowitz's L-15 medium (HyClone, Chicago, Ill.) supplemented with 10% fetal bovine serum (FBS).

The differences between covering the plates in parafilm in an incubator with 5% CO₂ to provide a CO₂-free environment versus placing plates without Parafilm M™ in a CO₂-free incubator were also examined, as commercial instructions for SW982 culture do not recommend CO₂ and air mixture culture conditions. Thus, all plates were covered in Parafilm M™ and placed into an incubator under culture conditions with 5% CO₂ with the exception of one plate, which was placed without Parafilm M™ in the incubator. An additional plate without Parafilm M™ was placed in an incubator that lacked CO₂. Randomization of groups was also examined in this example, where plates that were not randomized (i.e., each column represented the same condition) were compared to plates that had random assignment of conditions on the plate. Randomization was performed using a commercially available list randomizer, such as the randomizer available at random.org.

Following cell attachment for approximately 24±4 hours, cells were primed for 72 hours with assay media (AM) alone, or AM with activating factors. Assay media was 50% (v/v) complete growth medium in L-15 basal media. Activating factor comprised 1 ng/mL of IL-1β (Invitrogen catalog number RIL1BI, lot number U1287721A, Carlsbad, Calif.) and 2 ng/mL of TNF-α (Millipore catalog number GF023, lot number 2946036, Burlington, Mass.). After 72 hours, all media was exchanged for fresh media. Cells received either AM alone, AM with activating factor (IL-1β and TNF-α), or AM with activating factor in 100% or 50% ASA CM, prepared as described previously. After 48 hours following treatment, one well from each of the AM and activating factor groups representing each cell density condition were scored for confluency, with results shown in Table 2. In general, high passage cells grew faster and were more confluent than low passage cells, while plate randomization and Parafilm M™ coverings had little effect. However, cells incubated in an incubator lacking CO₂ resulted in higher confluency relative to those exposed to 5% CO₂ incubation conditions with or without Parafilm M™ coverings.

TABLE 2

| | Confluence | |
|---|---|---|
| Cell Density | Assay Media (AM) | Inflammation |
| 37,000 cells/well (“new”) | 50% confluent | 50% confluent |
| 25,000 cells/well (“new”) | 50% confluent | 50% confluent |
| 37,000 cells/well (“old”) | 80% confluent | 60% confluent |
| 25,000 cells/well (“old”) | 80% confluent | 60% confluent |
| 25,000 cells/well (“new”, | 50% confluent | 60% confluent |
| random) | | |
| 25,000 cells/well (“new”, | 90% confluent | 100% confluent |
| no CO₂) | | |
| 25,000 cells/well (“new”, | 50% confluent | 50% confluent |
| CO₂, no parafilm) | | |

Supernatant was collected after 48 hours in culture for analysis of TNF-α protein production, and plates were fixed with 4% paraformaldehyde (PFA) at 4° C. according to methods well known in the art. Supernatant was immediately used for ELISA to determine TNF-α concentration using a commercially available kit (DTAOOD, R&D Systems, Minneapolis, Minn.) according to the manufacturer's instructions.

Statistical analysis was performed using commercially available GraphPad Prism (GraphPad Software). A one-way analysis of variance (ANOVA) and a Durmett's multiple comparisons test was run. For all graphs, average±standard deviation is reported. * denotes p<0.05, ** denotes p<0.01, ***denotes p<0.001, and **** denotes p<0.0001. For all graphs, the solid bars represent 100% of the dose and the hashed bars represent 50% of the dose. The number of samples for all experiments is reported in the figure legends.

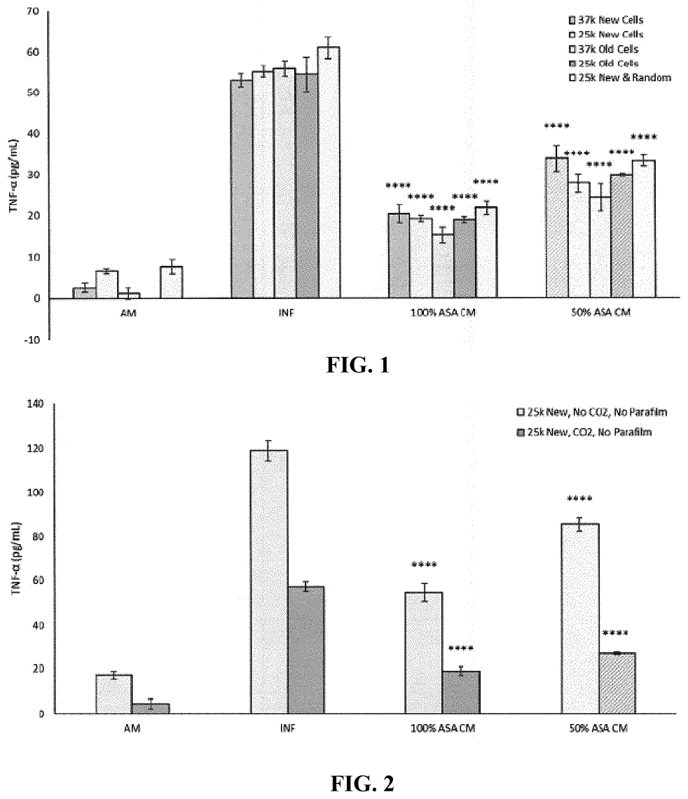

In comparing cell densities between the low passage treatment groups, results show that both densities tested preserve the significant trend of TNF-α downregulation following treatment with ASA CM (p<0.0001) and have similar TNF-α levels within each treatment group (FIG. 1). Furthermore, examination of the high passage treatment groups also shows preservation the significant trend (p<0.0001 for ASA CM compared to INF, activating factor) and both seeding densities result in TNF-α levels are similar within each treatment group. Both low passage and high passage treatment groups had similar TNF-α levels, and both treatment groups showed a significant reduction of TNF-α protein levels following treatment with ASA CM. For plate randomization studies, the significant downregulation of TNF-α is preserved following treatment with ASA CM (p<0.0001) and the TNF-α protein levels are similar to those produced in non-randomized plates. Thus, from the results in FIG. 1, it appears that passage number, seeding density, and plate randomization do not significantly affect the TNF-α levels, and similarly that plate randomization of well conditions does not significantly influence the TNF-α levels.

Next, the effects of incubation CO₂ conditions on TNF-α protein levels was investigated as shown by the results in FIG. 2. Cells were cultured in either an incubator without CO₂ or in flasks with a phenolic-like cap (no air exchange with the incubator). Once cells were seeded (25,000 cells/well) in 12 well plates, one plate was placed in a 5% CO₂ incubator (with no parafilm) and the other plate in an incubator with no CO₂. Examining the results comparing CO₂ and no CO₂, in both cases, treatment with ASA CM causes a significant decrease in TNF-α levels compared to activating factor (p<0.0001). While downregulation of TNF-α is observed as a result of treatment with ASA in both conditions, overall TNF-α levels were substantially higher in the plates that were maintained in the CO₂-free incubator. Furthermore, the condition of with CO₂ and Parafilm M™ coverings during incubation is shown in FIG. 1 (“25k New Cells” group). This group was found to have substantially equivalent TNF-α levels relative to those resulting from the 25,000 cells/well group incubated with CO₂ without Parafilm M™ coverings, as shown in FIG. 2. These results indicate the Parafilm M™ covers do not fully prevent interaction with CO₂ during incubation.

### Example 2

In this example, the effects of dosing SW982 cells with varying amounts of TNF-α at 10 ng/mL, 5 ng/mL, and 2 ng/mL is examined, while keeping the concentration of IL-1β constant at 1 ng/mL. SW982 cells at passage 29 were seeded at a density of 25,000 cells/well in 800 μL of growth media in four 12 well plates and allowed to attach overnight in an incubator without CO₂. Growth media was Leibowitz's L-15 medium (HyClone, Chicago, Ill.) supplemented with 10% fetal bovine serum (FBS).

Following attachment for approximately 24±4 hours, cells were primed for 72 hours with assay media (AM) alone, or assay media with activating factor. Assay media was 50% (v/v) complete growth medium in basal media (Leibowitz's L-15). The activating factor comprised 1 ng/mL of IL-1β (Invitrogen catalog number RIL1BI, lot number U1287721A, Carlsbad, Calif.) and either 10 ng/mL, 5 ng/mL, or 2 ng/mL of TNF-α (Millipore catalog number GF023, lot number 2946036, Burlington, Mass.). After 72 hours, all media was exchanged for fresh media. Cells received either AM alone, AM with activating factor (IL-1β at 1 ng/mL and TNF-α at 10 ng/mL, 5 ng/mL, or 2 ng/mL), or AM with activating factor (IL-1β at 1 ng/mL and TNF-α at 10 ng/mL, 5 ng/mL, or 2 ng/mL) in 100% or 50% ASA CM, prepared as described previously. Activating factor were independently added to both AM and CM immediately before use. Supernatant was collected after 48 hours in culture for analysis of TNF-α protein production, and plates were fixed with 4% PFA at 4° C. according to methods known in the art. Supernatant was stored at −80° C. until use in ELISAs. Levels of TNF-α were measured using a commercially available kit (DTAOOD, R&D Systems, Minneapolis, Minn.), and ELISAs were performed according to the manufacturer's instructions.

Statistical analysis was performed using commercially available GraphPad Prism (GraphPad Software). A one-way analysis of variance (ANOVA) and a Durmett's multiple comparisons test was run. For all graphs, average±standard deviation is reported. * denotes p<0.05, ** denotes p<0.01, ***denotes p<0.001, and **** denotes p<0.0001. For all graphs, the solid bars represent 100% of the dose and the hashed bars represent 50% of the dose. The number of samples for all experiments is reported in the figure legends.

The effect of activating factor on TNF-α responses was characterized to determine which TNF-α concentration would result in the greatest assay window between the AM control and the activating factor-alone groups. Additionally, the response to ASA CM at these varying concentrations of TNF-α treatment was characterized. The results of the TNF-α ELISA are shown in FIG. 3. For doses of 10 ng/mL of TNF-α, there was a high concentration of TNF-α in the activating factor-alone group and a significant reduction in TNF-α protein levels following treatment with ASA CM (100%: p<0.0001; 50%: p<0.001). For 5 ng/mL TNF-α treatment groups, there was a smaller response to TNF-α treatment, but still significant reductions in TNF-α protein levels in response to ASA CM (100%: p<0.0001; 50%: p<0.001). For 2 ng/mL TNF-α treatment groups, an even lower inflammatory response was observed, and a decrease in TNF-α in response to ASA CM (100%: p<0.01; 50%: n=1 and no statistical analysis) was also observed.

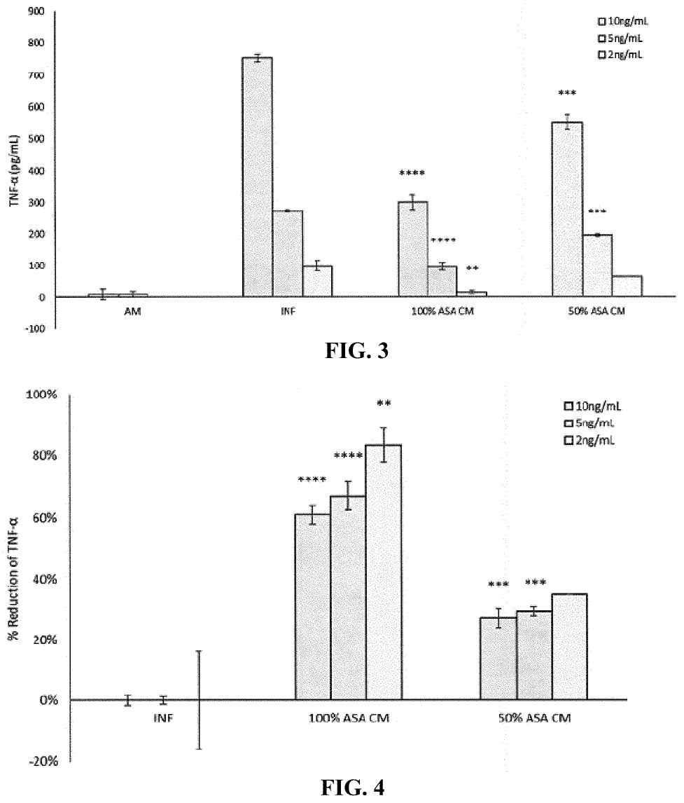

When ASA CM treatment was normalized and shown as percent reduction in FIG. 4 below, dosing with both 10 ng/mL and 5 ng/mL TNF-α resulted in very similar and significant reductions in inflammation following treatment with 100% and 50% ASA CM (p<0.0001 and p<0.001, respectively). In the 2 ng/mL treatment group, however, a trend for a higher percent reduction in TNF-α at 100% CM was observed while the overall inflammation levels were much lower than those observed in 10 ng/mL and 5 ng/mL TNF-α treatment groups (FIG. 3).

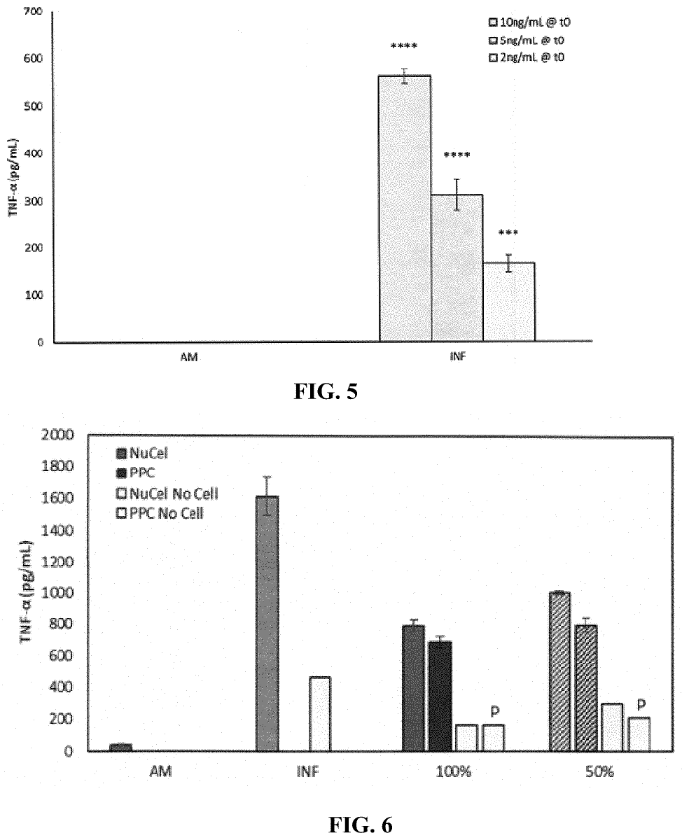

During this study, the initial priming of the SW982 cells was investigated to optimize increased levels of inflammation compared to AM controls, and to compare those levels to the inflammation levels observed at the end of the treatment (FIG. 3). The results from the TNF-α ELISA after 72 hours of priming with inflammatory cytokines (immediately before treatment, t0) is shown in FIG. 5. In the 10 ng/mL TNF-α treatment group, there was a significant increase in TNF-α levels at to compared to the AM control (p<0.0001). At t0, TNF-α levels in the 10 ng/mL TNF-α treatment group were 560 pg/mL (FIG. 5), while at the end of the experiment, TNF-α levels in the 10 ng/mL TNF-α treatment group were at 753 pg/mL (FIG. 3). In the 5 ng/mL TNF-α treatment group, there was a significant increase in TNF-α levels at t0 compared to AM control (p<0.0001), with values of 311 pg/mL (FIG. 5) at t0 and 273 pg/mL (FIG. 3) at the end of the study. In the 2 ng/mL TNF-α treatment group, there was a significant increase in TNF-α levels at t0 compared to AM control (p<0.001), with 164 pg/mL TNF-α at t0 (FIG. 5) and 99 pg/mL (FIG. 3) TNF-α at the end of the study. In the 10 ng/mL TNF-α group, the inflammation persists, as measured by TNF-α levels, and increases from t0 to the end of the study; in the 5 ng/mL and 2 ng/mL TNF-α groups, inflammation levels either remain constant or decrease from t0 to the final time point. Due to the preserved TNF-α reduction trends, the larger differences between the assay media and activating factor-alone groups, the reduction as a result of CM treatment groups, and the persistence/increase of TNF-α levels from t0 to the end of the experiment, it was determined that the use of 10 ng/mL of TNF-α with the SW982 cells was ideal.

### Example 3

The repeatability of the potency assay was investigated using a pooled positive control (PPC) and an ASA (NuCel®) CM from a commercial lot (lot number 184670903). Additionally, a dose curve was run to assess responses to different percentages of ASA CM. The PPC was created using NuCel commercial lots to make 5 batches of 30 vials each, ensuring that PPC CM was generated with roughly equal contributions from the various batches.

A flask of SW982 cells at passage 31 (passage+3 from thaw, 100% confluent) was used and seeded on one full 12-well plate and 8 wells of a second 12-well plate at 25,000 cells/well in growth media. For the dose curve, two additional 12-well plates were seeded at this cell density. All plates were placed into an incubator at 37° C. without CO₂. Growth media was Leibowitz's L-15 medium (HyClone, Chicago, Ill.) supplemented with 10% fetal bovine serum (FBS).

Following overnight attachment, one well from each of the AM and activating factor groups was scored for confluency, and cells were primed for 72 hours with AM alone, or AM with activating factor. AM was 50% (v/v) complete growth medium in basal media (Leibowitz's L-15). Activating factor comprised 1 ng/mL of IL-1β (Invitrogen catalog number RIL1BI, Carlsbad, Calif.) and 10 ng/mL of TNF-α (Millipore catalog number GF023, Burlington, Mass.). Activating factor was independently added to AM immediately before use. After 72 hours, one well from each of the AM and activating factor groups was scored for confluency and all media was exchanged for fresh media. Cells received either AM alone, AM with activating factor (IL-1β and TNF-α), or AM with activating factor in 100% or 50% PPC CM or ASA CM. Activating factor was independently added to both AM and CM immediately before use. For the dose curve, cells were treated with either 100%, 75%, 50%, 25%, or 10% ASA CM. Additionally, remaining media of approximately 400 μL from each treatment condition was placed into wells without cells. At approximately 48 hours following treatment, one well from each of the AM and activating factor groups was scored for confluency. The confluency of the cells in the AM and activating factor (INF) groups at each collected point are shown in Table 3.

TABLE 3

| | AM | INF |
|---|---|---|
| Initial Flask | Confluent | Confluent |
| Priming | 35% Confluent | 35% Confluent |
| Treatment | Overconfluent | Overconfluent |
| Collection | Overconfluent | Overconfluent |

The levels of TNF-α in the no cell controls, which consisted of remaining media left from each treatment group placed in empty wells and subject to the same conditions as the cells, are displayed in FIG. 6. These ELISA results show that all of the no cell control levels of TNF-α are substantially lower than their corresponding cell counterparts, indicating that that the cells themselves are producing TNF-α in excess of what the activating factor is delivering within each condition.

Supernatant was collected after approximately 48 hours in culture for analysis of TNF-α protein production. Supernatant was placed at −80° C. and stored until use in ELISAs. Levels of TNF-α were measured using a commercially available kit (DTAOOD, R&D Systems, Minneapolis, Minn.), and performed according to the manufacturer's instructions. All statistical analysis was undertaken using commercially available GraphPad Prism (GraphPad Software). A one-way analysis of variance (ANOVA) and a Dunnett's multiple comparisons test was run. For all graphs, average±standard deviation is reported. * denotes p<0.05, ** denotes p<0.01, *** denotes p<0.001, and **** denotes p<0.0001. For all graphs, the solid bars represent 100% dose and the hashed bars represent 50% dose.

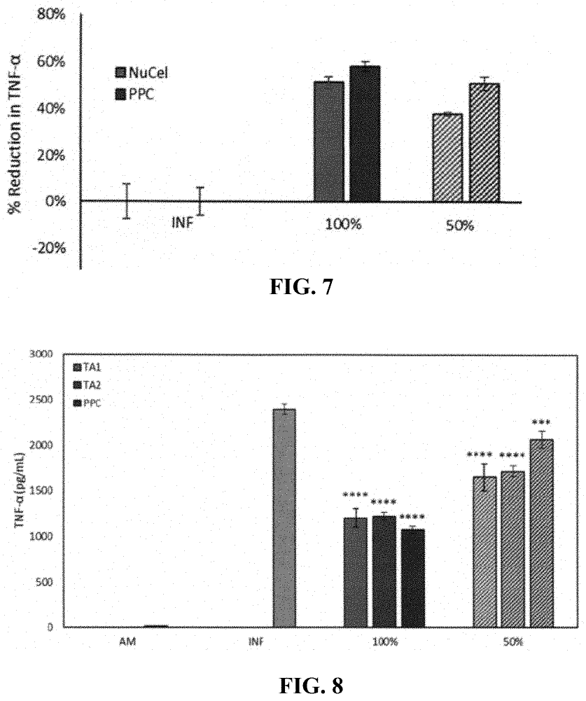

To assess the repeatability of the assay, percent reduction of inflammation is shown in FIG. 7. Once the experiments are normalized to their respective inflammation alone control values, the responses to treatment with 100% ASA CM and PPC CM and the response to 50% ASA CM and PPC CM are similar. Both 100% and 50% ASA CM and PPC CM groups saw the preservation of the decrease in TNF-α levels following administration, as well.

The repeatability of the assay was displayed in these studies. It was also confirmed that the cells produce excess TNF-α at levels greater than what is measured from the no cell controls, indicating that the TNF-α readouts are attributable to cell responses to activating factor and subsequent treatment, when applicable.

### Example 4

This study included a trial run of the potency assay using ASA CM generated from two clinical lots (TA1 and TA2) with PPC CM as a comparison and with one independent operator. Additionally, treatment with ASA CM from a NuCel commercial lot and PPC CM using an additional dose of 75% PPC CM was examined. Clinical lots and PPC vials were received in the lab and stored as recommended by the commercial vendor.

SW982 cells were used at passage 29 (passage+1 from thaw, <90% confluent). 12-well plates were seeded at 25,000 cells/well and placed into an incubator at 37° C. without CO₂. Growth media was Leibowitz's L-15 medium (HyClone, Chicago, Ill.) supplemented with 10% fetal bovine serum (FBS).

Following overnight attachment of approximately 24±4 hours, one well from each of the AM and activating factor groups were scored for confluency, and cells were primed for 72 hours with AM alone, or AM with activating factor. Assay media was 50% (v/v) complete growth medium in basal media (Leibowitz's L-15). Activating factor comprised 1 ng/mL of IL-1β (Invitrogen catalog number RIL1BI, Carlsbad, Calif.) and 10 ng/mL of TNF-α (Millipore catalog number GF023, Burlington, Mass.). Inflammatory cytokines were independently added to AM immediately before use. After 72 hours, one well from each of the AM and activating factor groups was scored for confluency and all media was exchanged for fresh media.

Cells received either AM alone, AM with activating factor (IL-1β and TNF-α), or AM with activating factor in 100% or 50% PPC CM or ASA CM. Inflammatory cytokines were independently added to both AM and CM immediately before use. For the dose curve, cells were treated with either 100%, 75%, or 50% ASA CM or PPC CM. Additionally, remaining media of approximately 800 μL from each treatment condition was placed into wells without cells. At approximately 48 hours following treatment, one well from each of the AM and activating factor groups was scored for confluency. The confluency of the cells in the AM and activating factor (INF) groups at each collected point are shown in Table 4.

TABLE 4

| | Operator 1 | | Operator 2 | |
|---|---|---|---|---|
| | AM | INF | AM | INF |
| Initial Flask | 70% | 70% | 90% | 90% |
| Priming | N/A | N/A | N/A | N/A |
| Treatment | 85% | 85% | 80% | 80% |
| Collection | Overconfluent | Overconfluent | Confluent | Confluent |

Supernatant was collected after approximately 48 hours in culture for analysis of TNF-α protein production. Supernatant was placed at −80° C. and stored until use in ELISAs. Levels of TNF-α were measured using a commercially available kit (DTAOOD, R&D Systems, Minneapolis, Minn.) and performed according to the manufacturer's instructions. Additionally, a Quantikine Immunoassay Control Group 248 (QC248, R&D Systems, Minneapolis, Minn.) was utilized. These controls were resuspended and run according to the manufacturer's instructions. A recovery and selectivity control were included to confirm that the TNF-α ELISA is capable of recovering TNF-α protein in the ASA CM. Standard TNF-α included in the kit was spiked into 50% CM at 500 pg/mL both alone (recovery control) and with 500 pg/mL IL-1β used for priming (selectivity control).

All statistical analysis was undertaken using commercially available GraphPad Prism (GraphPad Software). A one-way analysis of variance (ANOVA) and a Durmett's multiple comparisons test was run. For all graphs, average±standard deviation is reported. * denotes p<0.05, ** denotes p<0.01, ***denotes p<0.001, and **** denotes p<0.0001. For all graphs, the solid bars represent 100% dose and the hashed bars represent 50% dose.

A TNF-α ELISA was run to determine the response to inflammation following treatment with the two clinical lots (1904 and 1905) and with PPC CM as a control. These results are shown in FIG. 8. The addition of ASA CM from TAI, TA2, and PPC CM all result in a significant decrease in TNF-α levels following treatment (p<0.0001 for all except 50% PPC CM; p<0.001 for 50% PPC CM). These results are as expected with the reduced dose of CM resulting in less effective downregulation of TNF-α; additionally, PPC CM showed similar results as the test articles.

Next, 50%, 75%, and 100% CM doses were run using a commercial lot of ASA CM and the PPC CM; the results from the TNF-α ELISA are shown in FIG. 9. A significant decrease in TNF-α levels is observed following treatment with NuCel CM and PPC CM at 100%, 75%, and 50% doses (p<0.01 for all except 50% PPC CM; p<0.001 for 50% PPC CM). Additionally, there is a clear dose response observed, with greater levels of reduction seen with higher doses of CM.

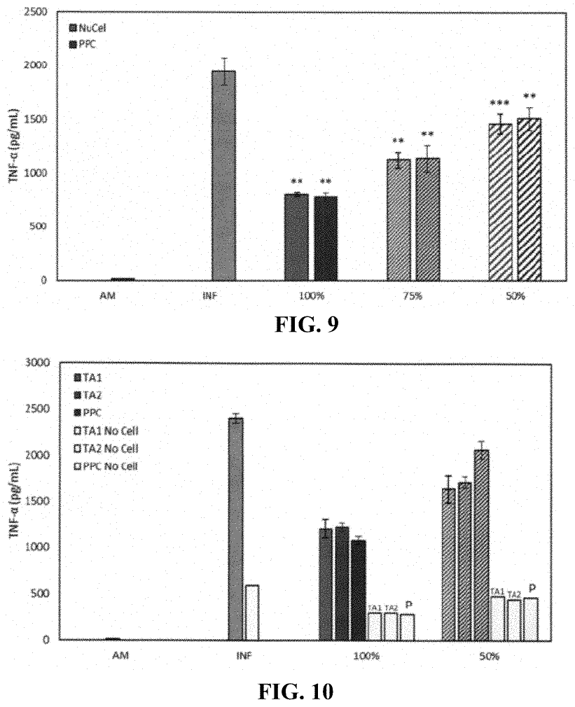

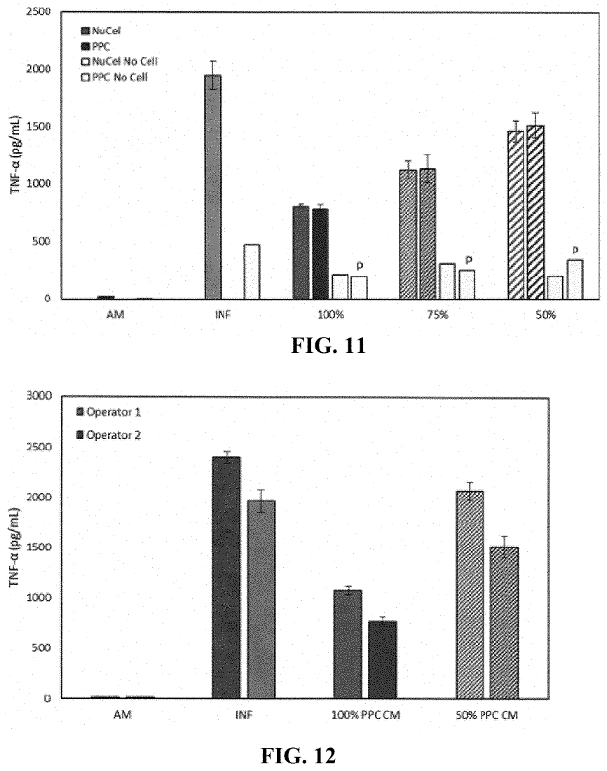

The levels of TNF-α in the no cell controls, which consisted of media from each treatment group, which was placed in empty wells and subjected to the same conditions as the cells, was examined. The results are shown in FIG. 10 and FIG. 11. The values from ASA CM from TA1 and TA2 and PPC CM for FIG. 10 are the same as shown in FIG. 8, and the values for ASA CM and PPC CM for FIG. 11 are the same as those shown in FIG. 9. For both FIGS. 10 and 11, all of the no cell control levels of TNF-α are lower than their corresponding cell counterparts. The results indicate that the cells themselves are producing TNF-α in excess of what the inflammation stimulation is delivering within each condition.

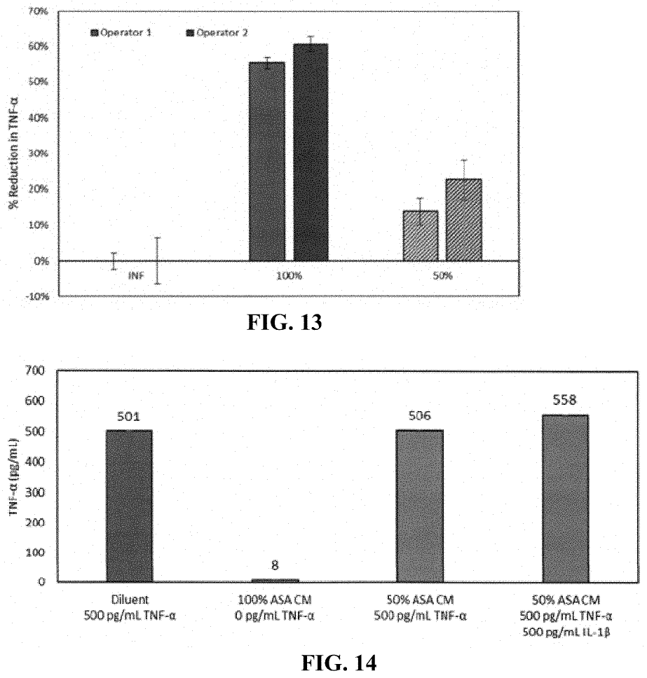

To assess the repeatability of the potency assay, FIG. 12 shows the results of the PPM CM from FIGS. 8 and 9 plotted on the same graph, while FIG. 13 shows the percent reduction in TNF-α for the results of PPM CM from FIGS. 8 and 9. In FIG. 12, the reduction in TNF-α levels following treatment with 100% and 50% PPC CM significant and similar between experiments. Comparing the coefficient of variance of the mean (CV) for each group, the activating factor group has a 14% CV, the 100% PPC CM group has a 23% CV, and the 50% PPC CM group has a 22% CV. In FIG. 13, normalizing the PPC CM results to inflammation results in similar values for both the 100% PPC CM and the 50% PPC CM groups, again highlighting the repeatability of the assay. The CV value for 100% PPC CM is 6%, while the 50% PPC CM has a value of 34%.

A study was undertaken to confirm that the TNF-α ELISA is capable of recovering TNF-α protein in the CM, as this is the active treatment. The results of the TNF-α levels are shown in FIG. 14. In FIG. 14, the 500 pg/mL TNF-α standard included with the kit was run in the diluent according to manufacturer's instructions. This returned a value of 501 pg/mL TNF-α. The 100% NuCel CM was run alone, without the addition of TNF-α, and measured 8 pg/mL of TNF-α. The recovery control, with 50% NuCel CM and 500 pg/mL TNF-α, returned a value of 506 pg/mL of TNF-α, while the selectivity control, which contained 50% NuCel CM, 500 pg/mL TNF-α, and 500 pg/mL of IL-1β, returned a value of 558 pg/mL TNF-α. These results confirm the ability of the TNF-α ELISA to accurately quantitate TNF-α in the CM both with and without IL-1β. Notably, ASA CM and NuCel CM are used interchangeably in this study.

In order to evaluate a single reportable metric for potency that could be used to compare relative potency between different experiments, percent reduction in TNF-α readouts at various doses were normalized to the PPC CM at the same dose to report relative potency at each dose and then averaged for a single readout. The relative potency for the 100%, 75%, and 50% ASA CM doses compared to the PPC CM are reported below in Table 5. Average, standard deviation, and percent coefficient of variance of the mean (CV) are reported. Based on this metric, the NuCel ASA lot would have reported potency of approximately 104±7%.

TABLE 5

| | Relative Potency | | |
|---|---|---|---|
| | Average | Standard | CV |
| 100% ASA CM | 98% | 4% | 4% |
| 75% ASA CM | 101% | 18% | 18% |
| 50% ASA CM | 111% | 32% | 29% |
| Average ASA CM | 104% | 7% | 7% |

This example confirmed the repeatability of the assay and that the two ASA clinical lots and the PPC CM preserved the significant decrease in TNF-α levels. The use of relative potency compared to PPC CM at three doses was also evaluated, including 75%. Additionally, the utility of the immunoassay control sets for the TNF-α assay was confirmed, as well as recovery and selectivity controls to confirm that the TNF-α ELISA is able to detect TNF-α protein in the CM. Finally, a relative potency value was calculated that can be used to compare across potency assays.

### Example 5

Three different models were examined to investigate the potency of ASA in down regulating inflammation. In this study, an in vitro model of OA was created by taking HFLS and placing the cells under an inflammatory stimulus. After a period of priming, these cells were then exposed to conditioned media from human ASA. Responses were then measured by determining protein levels in the supernatant of HFLS. Treatment with ASA resulted in decreased levels of pro-inflammatory cytokines and increased levels of anti-inflammatory cytokines.

Human recombinant IL-1β (Invitrogen) and TNF-α (Corning) were used at physiologically relevant levels for OA (1 ng/mL and 10 ng/mL, respectively). The HFLS (Cell Applications) were expanded using Synoviocyte Growth Medium (Cell Applications) according to manufacturer's protocols. All cells were used at doublings 4-6. CM was prepared by thawing ASA, centrifuging at 250×g for 5 minutes to remove the DMSO and resuspending the pellet in a volume of assay medium (75% Synoviocyte Basal Medium with no supplements and 25% Synoviocyte Growth Medium; low serum) equal to the original volume of ASA. The resuspended ASA was conditioned for 4 days at 4° C. on an orbital rocker to release most of the cytokines, diluted to 25% v/v in fresh assay medium and sterile filtered before use, leaving only the released proteins without any membrane or cell components.

To study the effects of ASA on the inflammatory synoviocyte model, normal HFLS were seeded at a density of 37,000 cells/well in a 12 well tissue culture plate and allowed to attach overnight under standard culture conditions. Cells were primed for 72 hours with assay medium (low levels of supplements) or assay medium with inflammatory cytokines (1 ng/mL human IL-1β and 10 ng/mL human TNF-α). After initial priming, all media were changed and replaced with fresh media and fresh inflammatory cytokines. Half of the wells received ASA CM plus fresh inflammatory cytokines, while the other half received assay media plus fresh inflammatory cytokines. After 48 hours of treatment, supernatant and cells were collected for the evaluation of protein levels and gene expression, respectively.

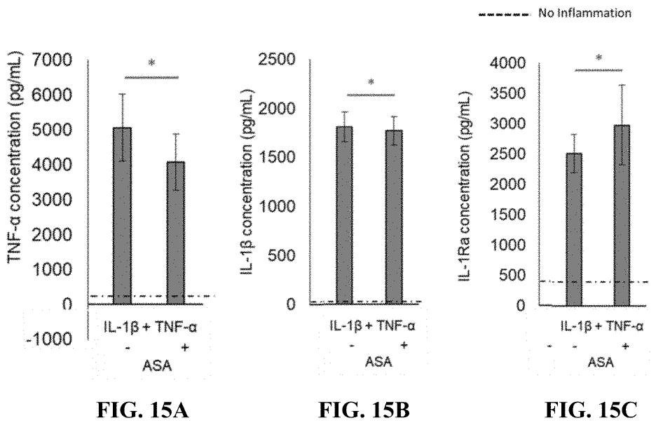

The supernatants were frozen at −80° C. until being assayed for IL-1β, TNF-α, IL-1Ra, and other relevant protein levels using ELISA kits (R&D Systems). Subsequent evaluation of representative conditioned media revealed low to undetectable levels of IL-1β and TNF-α and approximately 2 ng/mL of IL-1ra. Treatment with ASA resulted in significant decreases in pro-inflammatory cytokines, TNF-α and IL-1β, with the largest effect seen on TNF-α as shown in FIG. 15. There was also a significant increase seen in anti-inflammatory IL-1ra concentrations, as well as a small, but significant, increase in TIMP-3 levels (data not shown).

Overall, using an inflammatory model, it was determined that treatment of normal human fibroblast-like synoviocytes with ASA resulted in the downregulation of pro-inflammatory cytokine levels and the upregulation of anti-inflammatory cytokines and protease inhibitors.

Rabbit synoviocytes (HIG-82), are spontaneously immortalized rabbit synoviocytes that retain the activatable phenotype of primary rabbit synoviocyte cultures. The HIG-82 cell line was obtained from ATCC (Manassas, Va.) and cultured in Ham's F12 medium (Gibco, Grand Island, N.Y.), supplemented with 10% fetal bovine serum (Corning, Corning, N.Y.) and antibiotic-antimycotic solution (Corning, Corning, N.Y.). For the assay, the HIG-82 cells were seeded at a density of 37,000 cells/well in a 12-well plate and allowed to attach overnight. The same protocol described above in this Example 5 for the HFLS model was followed, however the cells were primed with 1 ng/mL rabbit IL-1β and 10 ng/mL rabbit TNF-α. ASA CM was prepared as for the HFLS model as described above in this Example 5. A total of 10 lots of ASA were evaluated in this model. Additionally, the separate contributions of the amnion membrane particles and cellular components were assessed, using three lots materials prepared in the lab from different donors and frozen prior to mixing the two components as is done for the clinical product.

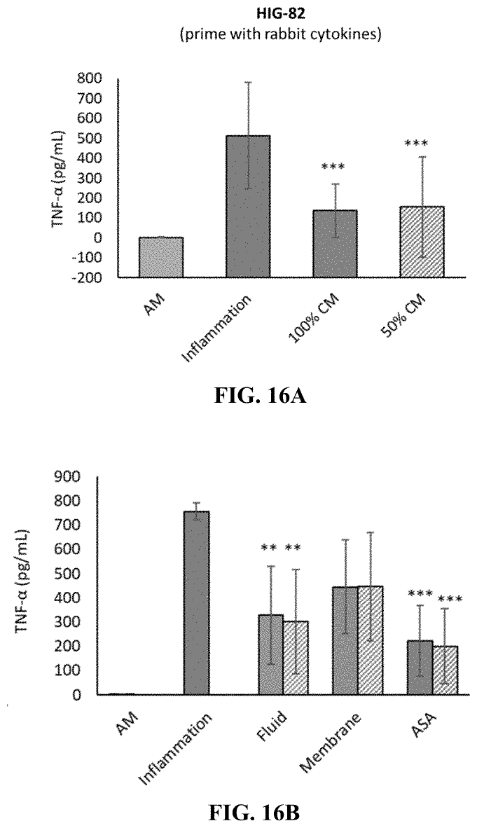

Multiple cytokines were analyzed in this model, but as for the HFLS model, the largest impact was seen on TNF-α levels (as shown in FIG. 16), with a significant decrease in protein expression seen with both a 50% and 100% dose of ASA-CM (conditioned medium), averaging a 79% decrease. Evaluating the individual components, a significant impact on TNF-α levels was seen with the fluid component alone.

Although some significant changes were seen in gene expression, most notably a decrease in IL-1β expression and an increase in MMP-1 expression, and an increase in protein expression of TIMP-1, no significant impact was seen in protein levels of IL-1β, MMP-1, and IL-1ra in this model.

The SW982 cell line was obtained from ATCC (Manassas, Va.) and cultured in Leibowitz's L-15 medium (HyClone, Chicago), supplemented with 10% fetal bovine serum (Corning, Corning N.Y.). L-15 is formulated to be used in the absence of CO₂, therefore, culture plates were wrapped in parafilm to reduce the exchange of gasses while being incubated in an incubator with 5% CO₂. For the assay, the SW982 cells were seeded at a density of 37,000 cells/well in a 12-well plate and all other methods were followed as for the HIG-82 model as disclosed above in this Example 5 with the exception that human IL-1β and TNF-α were used for priming.

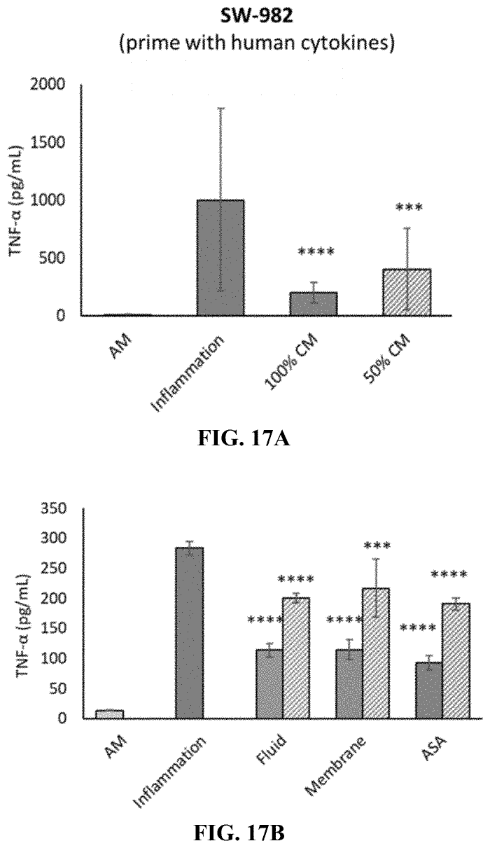

As with the HFLS and HIG-82 models, the largest impact was seen on TNF-α levels, as shown in FIG. 17. Compared to the HIG-82 cell line, significant decreases were seen with both the membrane and fluid components separately. In this model, a significant decrease in IL-1β gene expression, but conversely an increase in IL-1β protein levels was seen, and also a significant increase in IL-1ra protein levels.

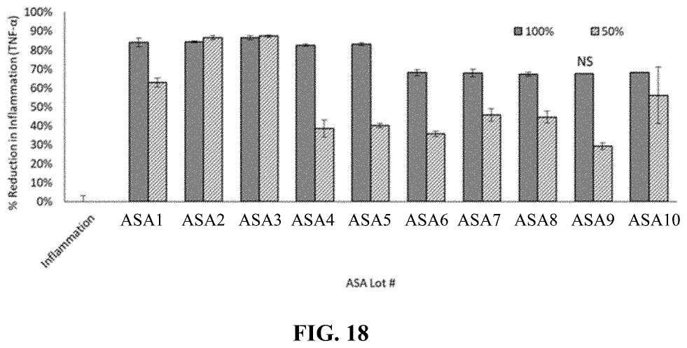

As can be noted by comparing the amount of TNF-α expression in the non-treated inflammation groups in FIG. 17, there was a variable inflammation response in separate experiments performed with this and the other models. To minimize the impact of this variability, the data can be normalized by calculating a percent reduction in TNF-α levels when compared to the inflammation control performed in the same experiment (as shown in FIG. 18). With this analysis, all 10 lots of ASA show a significant decrease when tested at the 50% CM dose and 9 of the 10 show a significant decrease at the 100% dose level (insufficient samples to perform statistics on lot 191570903). With many of the samples, there also was a dose-response seen with the average reduction of 77% (range 67-87%) with the 100% dose and an average reduction of 53% (range 29-87%) seen with the 50% dose.

The above results demonstrate that ASA impacted the balance of inflammation-associated factors, increasing the anti-inflammatory cytokines IL-1ra and TIMP-3 and decreasing the pro-inflammatory cytokines, TNF-α and IL-1β in a relevant human knee synoviocyte cell model. Similar responses, especially in the reduction of TNF-α levels, were seen with two transformed cell models, a spontaneously transformed rabbit synoviocyte cell line, HIG-82, and a human synovial sarcoma cell line, SW982, thus confirming that both cell lines retained the controllable inflammatory phenotype and can be used as in vitro cell-based methods to monitor ASA potency.

### Example 6

The inventors conducted two MSC inflammation characterization studies, with one run utilizing both bmMSCs and hTERT-adMSCs, and the second run utilizing just hTERT-adMSCs.

Primary bone marrow-derived MSCs (bmMSCs) and hTERT immortalized adMSCs (ASC52telo; hTERT-adMSCs) were obtained from ATCC (Manassas, Va.). Primary cells were routinely cultured in ATCC MSC basal medium supplemented with Mesenchymal Stem Cell Growth Kit for bmMSCs and passaged according to manufacturer's instructions. hTERT-adMSCs were routinely cultured in Dulbecco's Modified Eagle's Medium (DMEM) (Corning, Corning, N.Y.) supplemented with 10% fetal bovine serum (FBS) (Corning, Corning, N.Y.) and antibiotic-antimycotic solution (P/S/A) (Corning, Corning, N.Y.) and passaged according to manufacturer's instructions. DMEM+10% FBS+P/S/A served as the growth media (GM) for seeding experiments with both the primary and immortalized cells. supplemented with 5% FBS served as assay media (AM) control in all experiments. Conditioned media (CM) was prepared by thawing amniotic suspension allograft (ASA, Organogenesis, Birmingham, Ala.), followed by centrifugation at 600×g for 5 minutes to remove the DMSO component; the supernatant was removed and discarded. The pelleted component of ASA was then resuspended in AM at a concentration of 1 mL of AM per 1 mL of ASA (original volume) and incubated at 4° C. for ˜3 days with gentle rotation. The suspension was then centrifuged at 600×g for 5 minutes, and then the supernatant (CM) was collected and sterile filtered using a 0.22 μm polyethersulfone (PES) filter (Millex™-GP Sterile Syringe Filters, SLGP033RS, MilliporeSigma, Burlington, Mass.) and used undiluted (100%) or diluted in AM before use.

MSC response to two lots of ASA was evaluated using one primary lot of bmMSCs (70017992) and hTERT immortalized adMSCs (ASC52telo, hTERT-adMSCs). MSCs were seeded into 12-well plates at 20,000 cells/well and allowed to attach for 4-8 hours before being primed with 20 ng/mL TNF-α (GF023, Millipore, Burlington, Mass.) and 1 ng/mL IL-1β (RIL1B1, Invitrogen, Carlsbad, Calif.) to induce an inflamed phenotype. After approximately 72 hours of priming, cells were treated with either 100%, 75%, or 50% ASA CM (prepared as described above) with fresh inflammatory cytokines. Assay media controls (AM, which never receive inflammatory stimulus) and inflammation alone (INF, which received fresh cytokines but no ASA treatment after priming) serve as plate-wise controls. On the treatment day, remaining treatments from all conditions were added to an empty plate to serve as no cell controls, which enabled confirmation that observed responses are indeed cell mediated. Supernatant was collected after approximately 48 hours of treatment for analysis of TNF-α protein levels, and cells were collected in RNAzol (400 μL).

Supernatant was placed at −80° C. and stored until use in enzyme-linked immunosorbent assays (ELISAs). Levels of TNF-α were measured using commercially available kits (DTAOOD, R&D Systems, Minneapolis, Minn.). ELISAs were performed according to manufacturer's instructions.

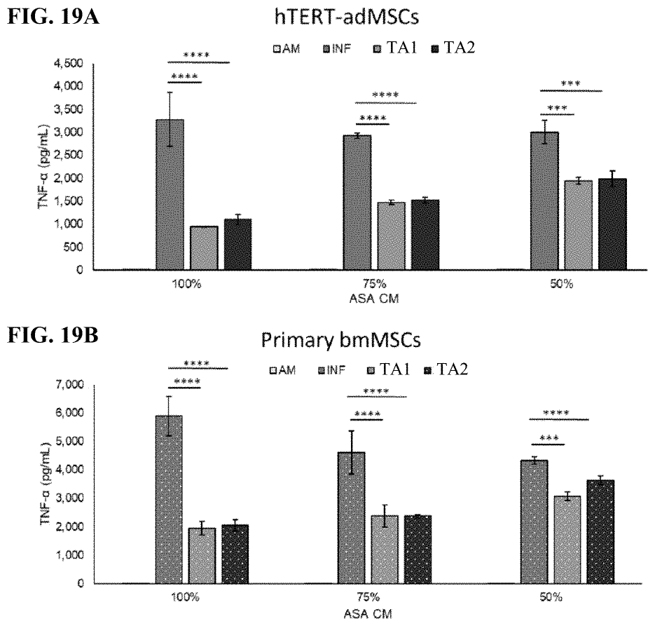

Treatment of hTERT-adMSCs and bmMSCs with ASA CM following priming with IL-1β and TNF-α resulted in significant decreases at the 100%, 75%, and 50% dose for both test articles (two ASA lots, denoted as TA1 and TA2, respectively) as shown in FIG. 19. All AM samples were below the lowest standard (15.6 pg/mL) and are reported as such. The average INF across all 3 plates for the hTERT-adMSCs was 3067.4 pg/mL with a CV of 6.0%, while the average INF for the primary bmMSCs was 4948.2 pg/mL with a CV of 16.8%.

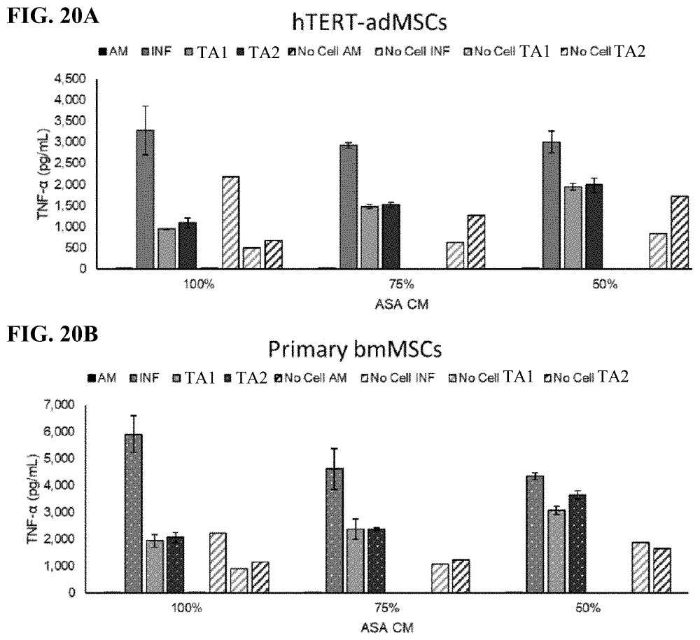

TNF-α levels in the no cell controls were also compared to cell supernatant samples to ensure that cells themselves are producing TNF-α in excess of what is delivered for each treatment condition. This data is shown in FIG. 20. The solid bars are the same as shown in FIG. 19 for comparison purposes, while the no cell controls are shown as hashed bars. Of note, no cell controls for AM and INF are only shown in the 100% group as the same stock is used to treat all dose plates.

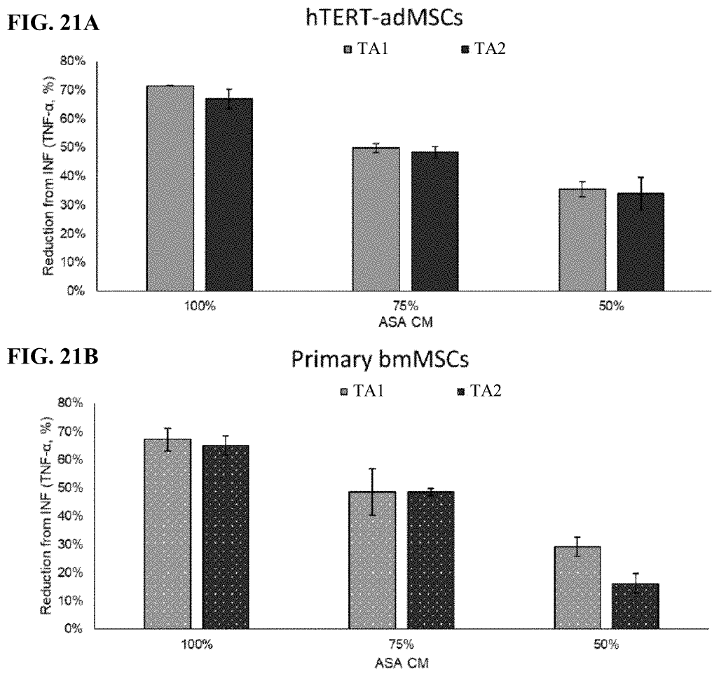

Percent reduction from INF was then calculated as described for each test article for each dose tested (as shown in FIG. 21). Both the TA1 and TA2 test articles resulted in a dose-dependent reduction in TNF-α levels at all doses.

Both hTERT-adMSCs and primary bmMSCs were able to significantly decrease levels of TNF-α at 100%, 75%, and 50% CM doses and in a dose dependent manner and produced levels of TNF-α in excess of what was being delivered in each condition. Furthermore, both cell types had a dose-dependent reduction in TNF-α levels at all doses.

MSC response to two lots of ASA was evaluated using hTERT immortalized adMSCs (ASC52telo, hTERT-adMSCs). MSCs were seeded into 12-well plates at 20,000 cells/well and allowed to attach for 4-8 hours before being primed with 20 ng/mL TNF-α (GF023, Millipore, Burlington, Mass.) and 1 ng/mL IL-1β (RIL1B1, Invitrogen, Carlsbad, Calif.) to induce an inflamed phenotype. After approximately 72 hours of priming, cells were treated with either 100%, 75%, or 50% ASA CM (prepared as described above) with fresh inflammatory cytokines. Assay media controls (AM, which never received inflammatory stimulus) and inflammation alone (INF, which received fresh cytokines but no ASA treatment after priming) serve as plate-wise controls. On the treatment day, remaining treatments from all conditions were added to an empty plate to serve as no cell controls, which enabled confirmation that observed responses are indeed cell mediated. Supernatant was collected after approximately 48 hours of treatment for analysis of TNF-α protein levels, and cells were collected in RNAzol (400 μL).

Supernatant was placed at −80° C. and stored until use in enzyme-linked immunosorbent assays (ELISAs). Levels of TNF-α were measured using commercially available kits (DTAOOD, R&D Systems, Minneapolis, Minn.). ELISAs were performed according to manufacturer's instructions.

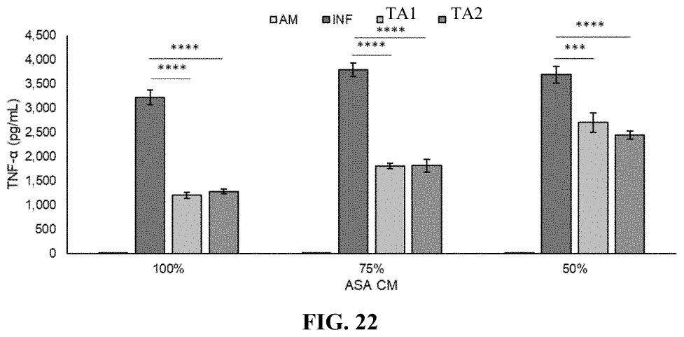

Treatment of hTERT-adMSCs with ASA conditioned media (CM) following priming with IL-1β and TNF-α resulted in significant downregulations at the 100%, 75%, and 50% dose for both test articles (ASA lots, denoted as TA1 and TA2, respectively) as shown in FIG. 22. All assay media (AM) samples were below the lowest standard (15.6 pg/mL) and are reported as such. The average INF across all 3 plates was 3560.9 pg/mL with a CV of 8.5%.

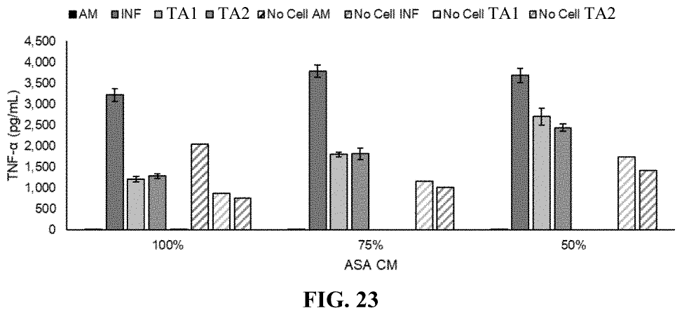

TNF-α levels in the no cell controls were also compared to treated cell samples to ensure that cells themselves are producing TNF-α in excess of what is delivered for each treatment condition. This data is shown in FIG. 23. The solid bars are the same as shown in FIG. 22 for comparison purposes, while the no cell controls are shown as hashed bars. Of note, no cell controls for AM and INF are only shown in the 100% group as the same stock is used to treat all dose plates.

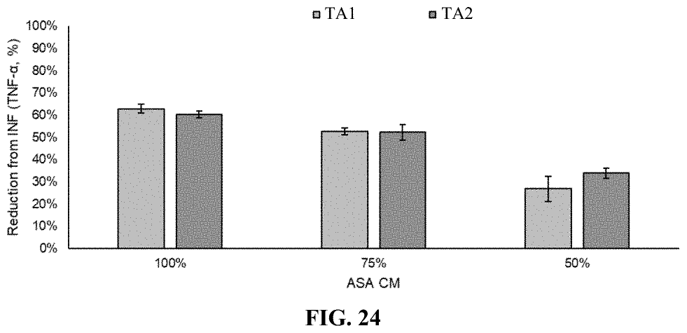

Percent reduction from INF was then calculated as described for each test article for each dose tested as shown in FIG. 24. Both the 1906 and 1908 test articles resulted in a dose-dependent reduction in TNF-α levels at all doses.

The results demonstrated that hTERT-adMSCs were able to significantly decrease levels of TNF-α at 100%, 75%, and 50% CM doses tested, and produced levels of TNF-α in excess of what was being delivered in each condition. Furthermore, hTERT-adMSCs resulted in a dose-dependent reduction in TNF-α levels at all doses.

Although the present invention and its advantages have been described in detail, it should be understood that various changes, substitutions and alterations can be made herein without departing from the spirit and scope of the invention as defined in the appended claims. The methods, assays, and the various embodiments thereof described herein are exemplary. Various other embodiments of the methods and assays described herein are possible.

What is claimed:

1. A method for quantitating an anti-inflammatory activity of a product, the method comprising:
adding the product or a substance derived from the product to an activated cell or tissue of a test system;
incubating the product or the substance derived from the product with the test system for a first period of time;
determining the extent the product or the substance derived from the product inhibits or stimulates the production, activity, or both the production and activity of a pro-inflammatory mediator from the activated cell or tissue to generate a product value; and
associating the product value with the product.

2. The method of claim 1 further comprising:
determining the extent the product or the substance derived from the product inhibits or stimulates the production, activity, or both the production and activity of an anti-inflammatory mediator to generate an additional product value; and
associating the additional product value with the product.

3. The method of claim 1, wherein the product is selected from the group consisting of:
an amnion-derived product; and an amnion suspension allograft.

4. The method of claim 1, wherein the substance derived from the product is selected from the group consisting of: a conditioned media generated from the product; and a factor isolated from a conditioned media generated from the product.

5. The method of claim 1, wherein the test system is an in vitro system comprising a cell line selected from the group consisting of: a mesenchymal stromal cell line; and a transformed human synovial cell line that retains an activatable phenotype.

6. The method of claim 1, further comprising the step of producing the activated cell or tissue by contacting the cell or tissue of the test system with an activating factor for a second period of time.

7. The method of claim 6, wherein the activating factor is selected from the group consisting of: IL-1β; TNF-α; and a combination of both IL-1β and TNF-α.

8. The method of claim 1, wherein the pro-inflammatory mediator is a cytokine selected from the group consisting of: IL-1α and IL-1β; TNF-α; IL-6; leukemia inhibitory factor; oncostatin-M; IL-8; IL-17; and IL-18.

9. The method of claim 2, wherein the anti-inflammatory mediator is a cytokine selected from the group consisting of: IL-4; IL-6; IL-10; IL-11; IL-13; IL-1 receptor antagonist (IL-1ra); IFN-γ; TGF-β; bone morphogenic protein; insulin-like growth mediator-I; and fibroblast growth mediator.

10. A method for standardizing an anti-inflammatory activity of a product, the method comprising:
adding the product or a substance derived from the product to an activated cell or tissue of a test system;
incubating the product or the substance derived from the product with the test system for a first period of time;
determining the extent the product or the substance derived from the product inhibits or stimulates the production, activity, or both the production and activity of a pro-inflammatory mediator from the activated cell or tissue to generate a product value;
comparing the product value with a reference value to determine a standardized value; and
associating the standardized value with the product.

11. The method of claim 10, wherein the reference value is determined by:
adding a standard to the activated cell or tissue of the test system;
incubating the standard with the test system for the first period of time; and
determining the extent the standard inhibits or stimulates the production, activity, or both the production and activity of the pro-inflammatory mediator from the activated cell or tissue to generate the reference value.

12. The method of claim 10 further comprising:
determining the extent the product or the substance derived from the product inhibits or stimulates the production, activity, or both the production and activity of an anti-inflammatory mediator to generate an additional product value;
comparing the additional product value with an additional reference value to determine an additional standardized value; and
associating the additional standardized value with the product,
wherein the additional standardized value is determined by: adding a standard to the activated cell or tissue of the test system; incubating the standard with the test system for the first period of time; and determining the extent the standard inhibits or stimulates the production, activity, or both the production and activity of the anti-inflammatory mediator from the activated cell or tissue to generate the additional standardized value.

13. The method of claim 10, wherein the product is selected from the group consisting of: an amnion-derived product; and an amnion suspension allograft.

14. The method of claim 10, wherein the substance derived from the product is selected from the group consisting of: a conditioned media generated from the product; and a factor isolated from a conditioned media generated from the product.

15. The method of claim 10, wherein the test system is an in vitro system comprising a cell line selected from the group consisting of: a mesenchymal stromal cell line; and a transformed human synovial cell line that retains an activatable phenotype.

16. The method of claim 10, further comprising the step of producing the activated cell or tissue by contacting the cell or tissue of the test system with an activating factor for a second period of time.

17. The method of claim 16, wherein the activating factor is selected from the group consisting of: IL-1β; TNF-α; and both IL-1β and TNF-α.

18. The method of claim 10, wherein the pro-inflammatory mediator is a cytokine selected from the group consisting of: IL-1α and IL-1β; TNF-α; IL-6; leukemia inhibitory factor; oncostatin-M; IL-8; IL-17; and IL-18.

19. The method of claim 12, wherein the anti-inflammatory mediator is a cytokine selected from the group consisting of: IL-4; IL-6; IL-10; IL-11; IL-13; IL-1 receptor antagonist (IL-1ra); IFN-γ; TGF-β; bone morphogenic protein; insulin-like growth mediator-I; and fibroblast growth mediator.

20. A method for determining, prior to administration of a product to a subject, whether administration of the product to the subject is likely to produce an anti-inflammatory effect in the subject, the method comprising:
adding the product or a substance derived from the product to an activated cell or tissue of a test system;
incubating the product or the substance derived from the product with the test system for a first period of time;
determining the extent the product or the substance derived from the product inhibits or stimulates the production, activity, or both the production and activity of a pro-inflammatory mediator from the activated cell or tissue to generate a product value;
comparing the product value with a reference value to determine a standardized value; and
comparing the standardized value to a threshold value, wherein when the standardized value is equal to or greater than the threshold value the anti-inflammatory effect is likely to be produced in the subject.

21. The method of claim 20, wherein the reference value is determined by:
adding a standard to the activated cell or tissue of the test system;
incubating the standard with the test system for the first period of time; and
determining the extent the standard inhibits or stimulates the production, activity, or both the production and activity of the pro-inflammatory mediator from the activated cell or tissue to generate the reference value.

22. The method of claim 20 further comprising:
determining the extent the product or the substance derived from the product inhibits or stimulates the production, activity, or both the production and activity of an anti-inflammatory mediator to generate an additional product value;
comparing the additional product value with an additional reference value to determine an additional standardized value, wherein when the additional standardized value is equal to or greater than a threshold value, the anti-inflammatory effect is likely to be produced in the subject.

23. The method of claim 22, wherein the additional reference value is determined by:
adding a standard to the activated cell or tissue of the test system;
incubating the standard with the test system for the first period of time; and
determining the extent the standard inhibits or stimulates the production, activity, or both the production and activity of the anti-inflammatory mediator from the activated cell or tissue to generate the additional reference value.

24. The method of claim 20, wherein the product is selected from the group consisting of: an amnion-derived product; and an amnion suspension allograft.

25. The method of claim 20, wherein the substance derived from the product is selected from the group consisting of: a conditioned media generated by incubating the product in a culture media; and a factor isolated from a conditioned media generated by incubating the product in a culture media.

26. The method of claim 20, wherein the test system is an in vitro system comprising a cell line selected from the group consisting of: a mesenchymal stromal cell line; and a transformed human synovial cell line that retains an activatable phenotype.

27. The method of claim 20, further comprising the step of producing the activated cell or tissue by contacting the cell or tissue of the test system with an activating factor for a second period of time.

28. The method of claim 27, wherein the activating factor is selected from the group consisting of: IL-1β; TNF-α; and both IL-1β and TNF-α.

29. The method of claim 20, wherein the pro-inflammatory mediator is a cytokine selected from the group consisting of: IL-1α and IL-1β; TNF-α; IL-6; leukemia inhibitory factor; oncostatin-M; IL-8; IL-17; and IL-18.

30. The method of claim 22, wherein the anti-inflammatory mediator is a cytokine selected from the group consisting of: IL-4; IL-6; IL-10; IL-11; IL-13; IL-1 receptor antagonist (IL-1ra); IFN-γ; TGF-β; bone morphogenic protein; insulin-like growth mediator-I; and fibroblast growth mediator.
